(19) United States
(12) Patent Application Publication
(10) Pub. No.: US 2021/0318336 A1
(43) Pub. Date: Oct. 14, 2021

(54) METHODS FOR TREATING AORTIC ANEURYSM DISEASE

(71) Applicants: THE TRUSTEES OF THE UNIVERSITY OF PENNSYLVANIA, Philadelphia, PA (US); Yale University, New Haven, CT (US)

(72) Inventor: Prashanth Vallabhajosyula, New Haven, CT (US)

(73) Assignees: THE TRUSTEES OF THE UNIVERSITY OF PENNSYLVANIA, Philadelphia, PA (US); Yale University, New Haven, CT (US)

(21) Appl. No.: 17/226,793

(22) Filed: Apr. 9, 2021

Related U.S. Application Data
(60) Provisional application No. 63/007,842, filed on Apr. 9, 2020.

(51) Int. Cl. G01N 33/68; C12Q 1/6883

(52) U.S. Cl. CPC G01N 33/6893; C12Q 1/6883; G01N 2333/70596; C12Q 2600/178; G01N 2800/329; C12Q 2600/158; G01N 2333/70525

(57) ABSTRACT

This present disclosure relates to the use of one or more biomarkers for diagnosis, screening, or monitoring aortic aneurysm disease (e.g., ascending aortic aneurysm, descending thoracic aortic aneurysm, abdominal aortic aneurysm and Marfan syndrome) in a biological sample (e.g., a blood sample) of a subject. Accordingly, this disclosure provides methods and kits for determining the presence of one or more biomarkers for aortic aneurysm disease in a biological sample of a subject; methods for using the presence of such biomarkers to predict or diagnose aortic aneurysm disease in a subject; and methods to select or modify a therapeutic regimen (e.g., a beta-blocker treatment) for a subject based on the use of such biomarkers.

### CROSS-REFERENCE TO RELATED APPLICATIONS

This application is a U.S. Patent Application which claims priority to U.S. Provisional Patent Application Ser. No. 63/007,842, filed on Apr. 9, 2020, the contents of which is incorporated by reference herein in its entirety.

### INTRODUCTION

The present disclosure provides techniques for treating an aneurysm using biomarkers of endothelial cell-derived microvesicles for predicting, diagnosing, and/or monitoring an aortic aneurysm disease in a subject.

### BACKGROUND

Aortic aneurysm disease remains a silent killer, with majority of patients dying from complications such as dissection and rupture of the aneurysm. An aortic aneurysm is an enlargement (dilation) of the aorta to greater than 1.5 times normal size. Aortic aneurysms can cause no symptoms except when ruptured. They are commonly located in the abdominal aorta but can also be located in the thoracic aorta. Aortic aneurysms cause weakness in the wall of the aorta and increase the risk of aortic rupture. When a rupture occurs, massive internal bleeding results and, unless treated immediately, shock and death can occur. Screening with ultrasound can be indicated in those at high risk, with treatment either by open or endovascular surgery. Worldwide it is estimated that in 2013, around 152,000 people died from aortic aneurysm disease.

Certain guidelines for screening and monitoring for aortic aneurysm disease are based on imaging techniques, rather than use of a noninvasive molecular marker for aortic aneurysm disease. Criteria for surgical treatment of aortic aneurysm can be based on aneurysm size and rate of dilation, and a proportion of patients develop acute emergencies such as aortic rupture and type A dissection before meeting the criteria for surgery.

Therefore, there remains a need for novel accurate non-invasive biomarker platforms for screening, diagnosis, and monitoring of aortic aneurysm disease.

### SUMMARY

The present disclosure provides methods of diagnosing and/or treating a subject with an aneurysm. An example method includes measuring, in a fraction of a biological sample from a subject, at least one biomarker; and administering an effective amount of an aneurysm inhibitor to the subject, when the at least one biomarker is reduced compared to a reference sample.

In certain embodiments, the fraction is enriched with endothelial cell-derived microvesicles. In certain embodiments, the endothelial cell-derived microvesicles comprise an endothelial cell specific protein. In certain embodiments, the endothelial cell specific protein is selected from the group consisting of VE-cadherin, ICAM-1, E-cadherin, endothelial nitric oxide synthetase, ECM1, ECM2, and combinations thereof.

In certain embodiments, the at least one biomarker is selected from the group consisting of VE-cadherin, ICAM-1, ECM1, ECM2, and combinations thereof. In certain embodiments, the at least one biomarker is a protein, a nucleic acid, a number of microvesicles, or combinations thereof.

In certain embodiments, the aneurysm is an aortic aneurysm. In certain embodiments, the aortic aneurysm is a descending aortic aneurysm, an ascending aortic aneurysm, and/or an abdominal aortic aneurysm.

In certain embodiments, the subject has Marfan syndrome.

In certain embodiments, the aneurysm inhibitor is selected from the group consisting of a beta blocker, a calcium channel blocker, an angiotensin II receptor blocker, a statin, and combinations thereof. In certain embodiments, the beta blocker is selected from the group consisting of acebutolol, atenolol, betaxolol, bisoprolol, carteolol, labetalol, metoprolol, nadolol, nebivolol, penbutolol, pindolol, propranolol, sotanol, timolol, and combinations thereof. In certain embodiments, the calcium channel blocker is selected from the group consisting of amlodipine, beprifil, diltiazem, felodipine, isradipine, nicardipine, nifedipine, nisoldipine, verapamil, and combinations thereof. In certain embodiments, the angiotensin II receptor blocker is selected from the group consisting of azilsartan, candesartan, eprosartan, irbesartan, losartan, olmesartan, termisartan, valsartan, and combinations thereof. In certain embodiments, the statin is selected from the group consisting of atorvastatin, fluvastatin, lovastatin, pitavastatin, pravastatin, rosuvastatin, simvastatin, and combinations thereof.

In certain embodiments, the present disclosure provides a kit for diagnosing and/or monitoring a subject with an aortic aneurysm, comprising reagents for detecting a marker specific for an endothelial cell-derived microvesicle. In certain embodiments, the kit comprises a packaged probe and primer set, arrays/microarrays, marker-specific antibodies or marker-specific antibody-conjugated beads or quantum dots. In certain embodiments, the kit comprises a pair of oligonucleotide primers, suitable for polymerase chain reaction or nucleic acid sequencing, for detecting the marker. In certain embodiments, the kit comprises a monoclonal antibody or antigen-binding fragment thereof, or a polyclonal antibody or antigen-binding fragment thereof, for detecting the marker. In certain embodiments, the marker specific for endothelial cell is selected from the group consisting of VE-cadherin, ICAM-1, E-cadherin, endothelial nitric oxide synthetase, ECM1, ECM2 and combinations thereof.

### BRIEF DESCRIPTION OF THE DRAWINGS

For a more complete understanding of the present disclosure and its features and advantages, reference is now made to the following description, taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts images of western blot analyses showing the initial non-normalized differences in the expression of both endothelial cell biomarkers, VE-cadherin and ICAM-1, in control and aneurysm patients. These values were normalized to TSG101 and plotted together in FIGS. 2A-2B and FIGS. 3A-3B.

FIGS. 2A and 2B depict analysis of the VE-cadherin expression. FIG. 2A depicts VE-cadherin protein expression values for 10 control patients and 10 aneurysm patients, which were categorized to show intergroup reliability. The data were normalized against TSG101.

FIG. 2B depicts VE-cadherin protein expression values for 10 control patients and 10 aneurysm patients and shows that VE-cadherin protein expression exhibits a universal downregulation in aneurysm patients as compared to control patients. The data were normalized to TSG101.

FIGS. 3A and 3B depict analysis of the ICAM-1 expression. FIG. 3A depicts ICAM-1 protein expression values for 10 control patients and 10 aneurysm patients, which were categorized to show intergroup reliability. The data were normalized to TSG101. FIG. 3B depicts ICAM-1 protein expression values for 10 control patients and 10 aneurysm patients and shows that ICAM-1 protein expression exhibits a universal downregulation in aneurysm patients as compared to control patients. The data were normalized to TSG101.

FIG. 4 depicts western blot analyses of VE-cadherin and ICAM-1 in fifteen sets of control and Marfan Syndrome mice. Endothelial cell markers VE-cadherin and ICAM-1 were significantly downregulated in the Marfan mice compared to controls. They were probed for exosome specific markers to confirm exosome analysis.

FIGS. 5A and 5B depict the analysis of the VE-cadherin expression. FIG. 5A depicts VE-cadherin protein expression values for 15 control mice and 15 Marfan mice, which were categorized to show intergroup reliability. The data were normalized to TSG101. FIG. 5B depicts VE-cadherin protein expression values for 15 control mice and 15 Marfan mice and shows that VE-cadherin protein expression exhibits a universal downregulation in the Marfan mice.

FIGS. 6A and 6B depict analysis endothelial cell marker in human plasma exosomes. FIG. 6A depicts images of western blot analyses of endothelial cell biomarkers, ECM1 and ECM2, in control and aneurysm patients, and shows that ECM1 and ECM2 are downregulated in aneurysm patients. FIG. 6B depicts images of western blot analyses of isolated ECM1-expressing endothelial cell-derived microvesicles from control and aneurysm patients. The number of ECM1-expressing endothelial cell-derived microvesicles were reduced in aneurysm patients compared to control patients.

FIGS. 7A-7C depict analysis endothelial cell marker in Marfan Syndrome plasma exosomes. FIG. 7A depicts western blot analyses of endothelial cell biomarkers, ECM1 and ECM2, in control and Marfan Syndrome mice. Endothelial cell markers were significantly downregulated in Marfan mice compared to controls. FIG. 7B depicts western blot analysis of ECM1-expressing endothelial cell-derived microvesicles in control and Marfan Syndrome mice. The number of ECM1-expressing endothelial cell-derived microvesicles were reduced in Marfan Syndrome mice compared to controls. FIG. 7C depicts the expression levels of ECM1 in control and Marfan Syndrome mice.

FIGS. 8A and 8B depict mRNA analysis of exosomes in patient plasma exosomes. FIG. 8A depicts ECM1 mRNA expression in tricuspid aortic valve (TAV) patients compared to control patients. ECM1 mRNA expression was significantly reduced in TAV patients. FIG. 8B depicts western blot analysis of ECM1-expressing endothelial cell-derived microvesicles in TAV patients and control patients. The number of ECM1-expressing endothelial cell-derived microvesicles were reduced in TAV patients compared to control.

FIG. 9 depicts a volcano plot of the differential gene expression data of miRNAs in aneurysm and control groups.

FIG. 10 depicts principal component analysis for clustering gene expression data of miRNAs in aneurysm and control groups.

FIG. 11 depicts table indicating the upregulated miRNAs and genes in aneurysm and control groups indicating a significant enrichment in angiogenesis pathways.

FIG. 12 depicts pathway analysis of the gene expression data observed in aneurysm and control groups.

FIG. 13 depicts a Venn diagrams summarizing differentially expressed genes.

FIGS. 14A-14D depict analysis of expression of both endothelial cell biomarkers, VE-cadherin and ICAM-1, in control and aneurysm patients. FIG. 14A shows a photograph of a plasma exosome. FIG. 14B shows quantification and size of exosomes. FIGS. 14C and 14D show representative western blotting of endothelial cell biomarkers.

FIGS. 15A-15D depict analysis of the VE-cadherin expression. The data were normalized against TSG101 expression.

FIG. 16A-16C depict mRNA analysis of exosomes in patient plasma exosomes. FIG. 16A depicts ECM1 mRNA expression in tricuspid aortic valve (TAV) patients compared to control patients. ECM1 mRNA expression was significantly reduced in TAV patients. FIG. 16B depicts western blot analysis of ECM1-expressing endothelial cell-derived microvesicles in TAV patients and control patients. FIG. 16C shows quantification of the data illustrated in FIGS. 16A-16B.

FIGS. 17A-17C depict effects on angiogenesis of endothelial specific EVs from MFS patients or controls.

FIGS. 18A-18E depict partial least squares regression (PLSR) modeling. A two-component model was trained using the top 50 RNA variables of importance for the model projection (VIPs). FIG. 18A shows scores plot of component 1 and 2 from PLSR analysis trained with 5 control and 5 aneurysm patients and top 50 VIP RNAs. FIG. 18B shows loadings plot of component 1 and 2 show VIP miRs and 1 piR covary with aneurysm size and VEcad levels. FIG. 18C shows PLSR model predictions of aneurysm size and VEcad levels correlated with observed measurements. FIG. 18D shows top 15 ranking miRs for aneurysm size and VEcad determined by the weighted coefficients for the PLSR model. FIG. 18E shows VIP miR gene targets determined using miRTarBase. 40/50 VIPs had validated targets. String-db was used to determine enriched miR target gene ontology (GO) biological process pathways.

FIG. 19 depicts expression of top 15 RNAs with the largest weighted coefficients for aneurysm size and VE-cadherin in the PLSR model.

FIG. 20 depicts a heatmap of patient-to-patient distances (maximum) using raw counts.

FIG. 21 depicts normalized log-CPM of top differentially expressed miRNAs in aneurysm and control groups.

FIG. 22 depicts a heatmap of filtered, log-transformed miRNA counts. Euclidean distances are with complete linkage.

FIG. 23 depicts a graphical representation of the sample processing for analysis of RNA cargoes.

FIG. 24 depicts a graphical representation of the data processing and filtering for analysis of RNA cargoes

FIG. 25 depicts PLSR model used for the analysis of RNA cargoes.

FIG. 26 depicts the pathway analysis used for the analysis of RNA cargoes.

FIG. 27 depicts expression levels of representative microRNAs.

### DETAILED DESCRIPTION

The present disclosure provides non-invasive methods related to the use of microvesicles, e.g., endothelial cell-derived microvesicles, to screen, diagnose, and/or monitor an aortic aneurysm or an aortic aneurysm disease in a subject. The present disclosure provides for methods and kits for determining the presence of one or more biomarkers for an aortic aneurysm in a biological sample of a subject, and methods for using the presence of such biomarkers to predict, diagnose and/or monitor an aortic aneurysm or an aortic aneurysm disease in a subject.

There is a critical need for the development of biomarker platforms for noninvasive diagnosis and monitoring of aortic aneurysms. Exosomes are tissue specific nanoparticles carrying protein and RNA cargoes that are released by many tissue types, including endothelial cells, in a condition specific manner into the circulation. The endothelial cellular pathophysiology associated with an aortic aneurysm would be reflected in their exosomes released into circulation. Therefore, profiling of plasma endothelial cell-derived exosomes and their cargoes would serve as a noninvasive biomarker for aortic aneurysm. For purposes of clarity of disclosure and not by way of limitation, the detailed description is divided into the following subsections:

1. Definitions;

2. Methods;

3. Biomarkers;

4. Microvesicle Isolation Techniques;

5. Protein Detection Techniques;

6. RNA Detection Techniques;

7. Kits; and

8. Exemplary Embodiments.

### 1. Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “biomarker” refers to a marker (e.g., an expressed gene, including mRNA, microvesicle pool profile, and/or protein) that allows detection of a disease in an individual, including detection of disease in its early stages. Early stage of a disease, as used herein, refers to the time period between the onset of the disease and the time point that signs or symptoms of the disease emerge. Biomarkers, as used herein, include microvesicles (e.g., exosomes), nucleic acid, and/or protein markers or combinations thereof. In certain non-limiting embodiments, the expression level of a biomarker as determined by mRNA and/or protein levels in a biological sample from an individual to be tested is compared with respective levels in a biological sample from the same individual, another healthy individual. In certain non-limiting embodiments, the presence or absence of a biomarker as determined by mRNA and/or protein levels in a biological sample from an individual to be tested is compared with the respective presence or absence in a biological sample from the same individual, or another healthy individual. In certain non-limiting embodiments, the presence or absence of a biomarker in a biological sample of a subject is compared to a reference control.

The terms “reference sample” or “reference,” as used interchangeably herein, refers to a control for a biomarker that is to be detected in a biological sample of a subject. For example, a control can be the level of a biomarker from a healthy individual free from aortic aneurysm disease. In certain embodiments, a control can be the level of a biomarker from a healthy individual that underwent treatment for an aortic aneurysm disease, wherein the healthy individual is non-symptomatic. In certain embodiments, a reference can be the level of a biomarker detected in a healthy individual that has never had the disease. In other embodiments, the reference can be a predetermined level of a biomarker that indicates presence aortic aneurysm in a subject. In other embodiments, the reference can be a predetermined level of a biomarker that indicates a subject is free from aortic aneurysm. In certain embodiments, the reference can be an earlier sample taken from the same subject.

As used herein, the term “aneurysm” refers to a bulging, weak area in the wall of a blood vessel. An aneurysm can occur in any blood vessel, but most often develops in an artery rather than a vein. An aneurysm can be categorized by its location, shape, and cause. For example, an aneurysm may be found in many areas of the body, such as brain (cerebral aneurysm), aorta (aortic aneurysm), neck, intestines, kidney, spleen, legs.

As used herein “aortic aneurysm disease” refers to any aortic aneurysm disease (e.g., abdominal aortic aneurysm disease, descending aortic aneurysm disease, and ascending aortic aneurysm disease) or aortic aneurysm associated disease (e.g., Marfan Syndrome disease) that a subject having such aortic aneurysm disease or aortic aneurysm associated disease (e.g. Marfan Syndrome disease) can develop a syndrome of aortic aneurysm at certain stage of the disease.

As used herein, the term “biological sample” refers to a sample of biological material obtained from a subject, e.g., a human subject, including a biological fluid, e.g., blood, plasma, serum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid, bronchoalveolar fluid, biliary fluid and combinations thereof. In certain non-limiting embodiments, the presence of one or more biomarkers is determined in a blood sample obtained from a subject. In certain non-limiting embodiments, the presence of one or more biomarkers is detected in a plasma sample obtained from a subject.

The term “patient” or “subject,” as used interchangeably herein, refers to any warm-blooded animal, e.g., a human. Non-limiting examples of non-human subjects include non-human primates, dogs, cats, mice, rats, guinea pigs, rabbits, fowl, pigs, horses, cows, goats, sheep, etc.

As used herein, the term “disease” refers to any condition or disorder that damages or interferes with the normal function of a cell, tissue, or organ.

An “effective amount” of a substance as that term is used herein is that amount sufficient to effect beneficial or desired results, including clinical results, and, as such, an “effective amount” depends upon the context in which it is being applied. An effective amount can be administered in one or more administrations.

As used herein, the term “aneurysm inhibitor” can be a molecule, e.g., chemical compound, that inhibits the growth and/or rupture of an aneurysm. An aneurysm inhibitor can reversibly or irreversibly inhibit the process involved in the growth and/or rupture of an aneurysm. In certain embodiments, an aneurysm inhibitor can reverse the presence of an aneurysm, e.g., aortic aneurysm.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more sign or symptoms, diminishment of extent of disease, stabilized (i.e., not worsening) state of disease, prevention of disease, delay or slowing of disease progression, and/or amelioration or palliation of the disease state. The decrease can be a 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

The term “microvesicle” as used herein, refers to vesicles that are released from a cell. In certain embodiments, the microvesicle is a vesicle that is released from a cell by exocytosis of intracellular multivesicular bodies. In certain embodiments, the microvesicles can be exosomes. In certain embodiments, the microvesicles can be in the size range from about 30 nm to 1000 nm.

The term “endothelial cell-derived microvesicles,” as used herein, refers to microvesicles that are derived from endothelial cells.

### 2. Methods

The present disclosure provides methods for screening, diagnosing, treating and/or monitoring a subject with an aortic aneurysm by analyzing microvesicles released from endothelial cells. In certain embodiments, the present disclosure provides methods for isolating, detecting, purifying and/or analyzing microvesicles derived from endothelial cells (“endothelial cell-derived microvesicles”) to diagnosis a subject with an aortic aneurysm and/or monitor a subject that has an aortic aneurysm. In certain embodiments, such isolation is accomplished via microvesicle selection based on the presence of one or more microvesicle surface proteins. In certain embodiments, the methods of the present disclosure can further include the detection and/or analysis of one or more biomarkers associated with the endothelial cell-derived microvesicles, e.g., exosomes. The biomarkers that can be used in the present disclosure are set forth below.

In certain embodiments, the information provided by the methods described herein can be used by the physician in determining the most effective course of treatment (e.g., preventative or therapeutic). A course of treatment refers to the measures taken for a patient after the diagnosis of aortic aneurysm is made. For example, when a subject is identified to have an aortic aneurysm or is at risk of having an aortic aneurysm, the physician can determine whether frequent monitoring of endothelial cell-derived microvesicles and/or biomarkers associated with such microvesicles is required as a prophylactic measure.

In certain embodiments, the endothelial cell-derived microvesicles can be exosomes. In certain embodiments, the microvesicles can be in the size range from about 30 nm to 1000 nm. For example, and not by way of limitation, the microvesicles can be from about 30 nm to about 900 nm, from about 30 nm to about 800 nm, from about 30 nm to about 700 nm, from about 30 nm to about 600 nm, from about 30 nm to about 500 nm, from about 30 nm to about 400 nm, from about 30 nm to about 300 nm, from about 30 nm to about 200 nm, from about 30 nm to about 100 nm or from about 30 nm to about 50 nm in size. In certain embodiments, microvesicles can have a size range from about 30 nm to 200 nm. In certain embodiments, microvesicles can have an average size less than about 200 nm.

In certain embodiments, and as noted above, methods for assessing whether a subject suffers from aortic aneurysm and/or the isolation and/or purification of endothelial cell-derived microvesicles from a subject include obtaining at least one biological sample from the subject. In certain embodiments, the microvesicles can be detected in blood (including plasma or serum). The step of collecting a biological sample can be carried out either directly or indirectly by any suitable technique. For example, and not by way of limitation, a blood sample from a subject can be carried out by phlebotomy or any other suitable technique, with the blood sample processed further to provide a serum sample or other suitable blood fraction for analysis.

In certain embodiments, the aortic aneurysm can be an abdominal aortic aneurysm. In certain embodiments, the aortic aneurysm can be an ascending aortic aneurysm. In certain embodiments, the aortic aneurysm can be a descending aortic aneurysm. In certain embodiments, the aortic aneurysm disease can be Marfan Syndrome (MFS). In certain embodiments, the aortic aneurysm disease can be Loeys-Dietz Syndrome (LDS). In certain embodiments, the aortic aneurysm can be Ehlers-Danlos Syndrome (EDS). In certain embodiments, the aortic aneurysm can be a Familial Thoracic Aortic Aneurysm and/or Dissection (FTAAD).

Furthermore, the effectiveness of a medical therapy, e.g., an administration of beta blockers or angiotensin receptor blockers, for a patient having an aortic aneurysm can be monitored by evaluating the presence and/or levels of the one or more biomarkers over the course of a therapy, and decisions can be made regarding the type, duration and course of therapy based on these evaluations.

Non-limiting embodiments of the invention are described by the present specification and Examples.

2.1. Diagnostic and Monitoring Methods

In certain embodiments, a method for diagnosing a subject with an aortic aneurysm is disclosed. In certain embodiments, the method can include (a) obtaining a biological sample from the subject; (b) isolating one or more biomarkers from the biological sample; and (c) diagnosing an aortic aneurysm in the subject, wherein the presence and/or change in the level of the one or more biomarkers indicates the presence of the aortic aneurysm in the subject. For example, but not by way of limitation, the biomarker can be endothelial cell-derived microvesicles obtained from the biological sample. In certain embodiments, a change in the size, number and/or concentration of the isolated endothelial cell-derived microvesicles indicates the presence of an aortic aneurysm in the subject. In certain embodiments, the biomarker can be a biomarker, e.g., protein or nucleic acid (e.g., mRNA), present on the surface or within the microvesicles.

In certain embodiments, a method for diagnosing a subject with an aortic aneurysm can include: (a) obtaining a biological sample from the subject; (b) isolating one or more endothelial cell-derived microvesicles from the biological sample; (c) determining the presence and/or level of one or more biomarkers associated with the isolated endothelial cell-derived microvesicles; and (d) diagnosing an aortic aneurysm in the subject, wherein the change in the presence and/or level of the one or more biomarkers is diagnostic of an aortic aneurysm in the subject.

In certain embodiments, a method for diagnosing a subject with an aortic aneurysm can include: (a) obtaining a biological sample from the subject; (b) isolating one or more endothelial cell-derived microvesicles from the biological sample; (c) determining the presence and/or level of an endothelial cell specific protein associated with the isolated endothelial cell-derived microvesicles and/or determining the number of endothelial cell specific protein-expressing endothelial cell-derived microvesicles in the biological sample; and (d) diagnosing an aortic aneurysm in the subject, wherein a reduction in level of endothelial cell specific protein associated with the endothelial cell-derived microvesicles and/or a reduction in the number of endothelial cell specific protein-expressing endothelial cell-derived microvesicles in the biological sample as compared to a reference control is diagnostic that the subject has an aortic aneurysm.

In certain embodiments, a level of endothelial cell specific protein expression associated with the endothelial cell-derived microvesicles that is less than about 0.75, e.g., less than about 0.5, less than about 0.4, less than about 0.3 or less than about 0.2 as compared to a reference control is diagnostic that the subject has an aortic aneurysm. In certain embodiments, the endothelial cell specific protein is selected from the group consisting of VE-cadherin, ICAM-1, E-cadherin, endothelial nitric oxide synthetase, ECM1, ECM2 and combinations thereof. In certain embodiments, the endothelial cell specific protein is VE-cadherin, ICAM-1 or ECM1.

In certain embodiments, a level of endothelial cell mRNA expression associated with the endothelial cell-derived microvesicles that is less than about 0.75, e.g., less than about 0.5, less than about 0.4, less than about 0.3 or less than about 0.2 as compared to a reference control is diagnostic that the subject has an aortic aneurysm. In certain embodiments, the endothelial cell specific mRNA is selected from the group consisting of mRNAs encoding VE-cadherin, ICAM-1, E-cadherin, endothelial nitric oxide synthetase, ECM1, ECM2 and combinations thereof. In certain embodiments, the endothelial specific mRNA is an mRNA encoding VE-cadherin, ICAM-1 or ECM1.

In certain embodiments, a decrease of at least about 1.5 times, at least about 2 times, at least about 2.5 times, at least about 3 times, at least about 3.5 times, at least about 4.0 times, at least about 4.5 times or at least about 5 times in the number of endothelial cell-derived microvesicles that express a biomarker as compared to a reference sample is indicative that the subject has aortic aneurysm. In certain embodiments, a decrease of at least about 2 times the number of endothelial cell-derived microvesicles that express a biomarker as compared to a reference sample is diagnostic that the subject has aortic aneurysm.

The present disclosure further discloses that the size of the aneurysm is associated with the level of the biomarkers (e.g., VE-cadherin level, ECM2, ECM1 and/or ICAM-1 level in endothelial-specific exosomes). As such, the present disclosure further provides a method for monitoring the size of an aneurysm in a subject, comprising (a) obtaining a biological sample from the subject; (b) isolating, purifying, and/or identifying one or more endothelial cell-derived microvesicles from the biological sample; (c) detecting the presence or level of one or more biomarkers from the isolated, purified, or identified endothelial cell-derived microvesicles, wherein the change in the level of the one or more biomarkers indicates the size of the aneurysm in the subject has changed. In certain embodiments, a decrease in the level of the one or more biomarkers (e.g., VE-cadherin level, ECM1 level and/or ICAM-1 level in endothelial-specific exosomes) indicates that the size of the aneurysm in the subject has increased. In certain embodiments, an increase in the level of the one or more biomarkers indicates that the size of the aneurysm in the subject has decreased.

The present disclosure further provides methods for monitoring aortic aneurysm in a subject that is at risk of aortic aneurysm. In certain embodiments, the method can include determining the level of one or more biomarkers in a biological sample obtained from the subject subsequent to a diagnosis of aortic aneurysm and determining the presence or level of the one or more biomarkers in a biological sample obtained from the subject at one or more later timepoints. In certain embodiments, a change in the level of the one or more biomarkers in the second or subsequent samples, relative to the first sample indicates that there is a change in the severity of the aortic aneurysm of the subject.

In certain embodiments, the present disclosure further provides methods for monitoring a subject at risk of developing an aortic aneurysm. In certain embodiments, a subject at risk of developing an aortic aneurysm is an individual that suffered from an aortic aneurysm before. For example, and not by way of limitation, the method can include determining the level of one or more biomarkers in a biological sample obtained from the subject prior to a diagnosis of aortic aneurysm and determining the presence or level of the one or more biomarkers in a biological sample obtained from the subject at one or more later timepoints. In certain embodiments, a change in the level of the one or more biomarkers in the second or subsequent samples, relative to the first sample can indicate that the subject has developed aortic aneurysm disease.

In certain embodiments, the one or more biomarkers can be detected in blood (including plasma or serum) or in urine, or alternatively at least one biomarker can be detected in one sample, e.g., the blood, plasma or serum, and at least one other biomarker is detected in another sample, e.g., in urine. The collecting a biological sample can be carried out either directly or indirectly by any suitable technique. For example, a blood sample from a subject can be carried out by phlebotomy or any other suitable technique, with the blood sample processed further to provide a serum sample or other suitable blood fraction.

In certain embodiments, the aortic aneurysm disease is an abdominal aortic aneurysm disease. In certain embodiments, the aortic aneurysm disease is an ascending aortic aneurysm disease. In certain embodiments, the aortic aneurysm disease is an MFS disease. Exemplary biomarkers that can be used in the methods of the present disclosure are presented below.

2.2. Methods of Treatment

The present disclosure further provides methods of treating a subject with an aortic aneurysm. In certain embodiments, the method can include diagnosing a subject with an aortic aneurysm as disclosed herein, followed by the treatment of the subject. For example, but not by way of limitation, a method of treatment can include: (a) obtaining a biological sample from a subject; (b) isolating one or more biomarkers from the biological sample; (c) diagnosing the subject with an aortic aneurysm when there is a change in the presence and/or level of the one or more biomarkers as compared to a reference control level; and (d) treating the subject diagnosed with an aortic aneurysm. Non-limiting exemplary treatments of an aortic aneurysm include surgery removal, administration of beta blocker (e.g., metoprolol (Lopressor, Toprol-XL), atenolol (Tenormin) and bisoprolol (Zebeta)), angiotensin II receptor blockers (e.g., losartan (Cozaar), valsartan (Diovan) and olmesartan (Benicar)), and/or statins (atorvastatin (Lipitor), lovastatin (Altoprev), simvastatin (Zocor)).

In certain embodiments, the methods disclosed herein can be used to monitor the response in a subject to prophylactic or therapeutic treatment (for example, treatment for aortic aneurysm, as disclosed above). For example, but not by way of limitation, the disclosed subject matter further provides a method of treatment including measuring the presence and/or level of one or more biomarkers of the present disclosure in a subject at a first time point, administering a therapeutic agent, re-measuring the one or more biomarkers at a second time point, comparing the results of the first and second measurements and optionally modifying the treatment regimen based on the comparison. In certain embodiments, the first time point is prior to an administration of the therapeutic agent, and the second time point is after the administration of the therapeutic agent. In certain embodiments, the first time point is prior to the administration of the therapeutic agent to the subject for the first time. In certain embodiments, the dose (defined as the quantity of therapeutic agent administered at any one administration) is increased or decreased in response to the comparison. In certain embodiments, the dosing interval (defined as the time between successive administrations) is increased or decreased in response to the comparison, including total discontinuation of treatment.

Additionally, the method of the present disclosure can be used to determine the efficacy of a disease therapy, wherein a change in the level and/or presence of a biomarker in a biological sample of a subject can indicate that the therapy regimen can be increased, maintained, reduced or stopped.

In certain embodiments, the method of treating can include measuring, in a fraction of a biological sample from a subject, at least one biomarker; and administering an effective amount of an aneurysm inhibitor to the subject, when the at least one biomarker is reduced compared to a reference sample. In certain embodiments, the method of treating can include measuring, in a fraction of a biological sample from a subject, at least one biomarker; and administering an effective amount of an aneurysm inhibitor to the subject, when the at least one biomarker is increased compared to a reference sample. In certain embodiments, the fraction is enriched with exosomes and/or microvesicles. In certain embodiments, the fraction is prepared by isolating microvesicles from about 30 nm to about 200 nm in size. In certain embodiments, the fraction is prepared by purifying the microvesicles using an antibody binding to an endothelial cell-derived protein. For example, but without any limitation, the endothelial cell-derived protein can be one or more of ACE/CD143, C1qR1/CD93, VE-Cadherin, CC Chemokine Receptor D6, CD31/PECAM-1, CD34, CD36/SR-B3, CD151, CD160, CD300g/Nepmucin, CL-K1/COLEC11, CL-P1/COLEC12, Coagulation Factor III/Tissue Factor, DC-SIGNR/CD299, DCBLD2/ESDN, ECSCR, EMMPRIN/CD147, Endoglin/CD105, Endomucin, Endosialin/CD248, EPCR, Erythropoietin R, ESAM, FABPS/E-FABP, FABP6, ICAM-1/CD54, ICAM-2/CD102, IL-1 RI, IL-13 R alpha 1, Integrin alpha 4/CD49d, Integrin alpha 4 beta 1, Integrin alpha 4 beta 7/LPAM-1, Integrin beta 2/CD18, KLF4, LYVE-1, MCAM/CD146, Nectin-2/CD112, PD-ECGF/Thymidine Phosphorylase, Podocalyxin, Podoplanin, S1P1/EDG-1, S1P2/EDG-5, S1P3/EDG-3, S1P4/EDG-6, S1P5/EDG-8, E-Selectin/CD62E, E-Selectin (CD62E)/P-Selectin (CD62P), P-Selectin/CD62P, SLAM/CD150, Stabilin-1, Stabilin-2, TEM7/PLXDC1, TEM8/ANTXR1, Thrombomodulin/BDCA-3, THSD1, THSD7A, Tie-2, TNF RI/TNFRSF1A, TNF RII/TNFRSF1B, TRA-1-85/CD147, TRAILR2/TNFRSF10B, TRAILR1/TNFRSF10A, VCAM-1/CD106, VE-Statin, VEGFR1/Flt-1, VEGFR2/KDR/Flk-1, VEGFR3/Flt-4, VG5Q, vWF-A2. In certain embodiments, the aneurysm inhibitor is selected from the group consisting of beta blockers, calcium channel blockers, angiotensin II receptor blockers, statins, and combinations thereof.

In certain embodiments, the beta blocker is selected from the group consisting of acebutolol, atenolol, betaxolol, bisoprolol, carteolol, labetalol, metoprolol, nadolol, nebivolol, penbutolol, pindolol, propranolol, sotanol, timolol, and combinations thereof. In certain embodiments, the calcium channel blocker is selected from the group consisting of amlodipine, beprifil, diltiazem, felodipine, isradipine, nicardipine, nifedipine, nisoldipine, verapamil, and combinations thereof. In certain embodiments, the angiotensin II receptor blocker is selected from the group consisting of azilsartan, candesartan, eprosartan, irbesartan, losartan, olmesartan, termisartan, valsartan, and combinations thereof. In certain embodiments, the statin is selected from the group consisting of atorvastatin, fluvastatin, lovastatin, pitavastatin, pravastatin, rosuvastatin, simvastatin, and combinations thereof.

In certain embodiments, the aneurysm inhibitor can be administered to a subject at a dose of about 0.05 mg/kg to about 100 mg/kg. In certain embodiments, a subject can be administered up to about 2,000 mg of the aneurysm inhibitor in a single dose or as a total daily dose. For example, but not by way of limitation, a subject can be administered up to about 1,800 mg, up to about 1,500 mg, 1,200 mg, up to about 1,000 mg, up to about 800 mg, up to about 500 mg, up to about 200 mg, up to about 150 mg, up to about 100 mg, up to about 50 mg or up to about 25 mg of the aneurysm inhibitor in a single dose or as a total daily dose. It is to be understood that, for any particular subject, specific dosage regimes should be adjusted over time according to the individual need, the expression levels of a biomarker, and/or the professional judgment of the person administering or supervising the administration of the aneurysm inhibitor. For example, the dosage of the aneurysm inhibitor can be increased if the lower dose does not provide sufficient activity in the treatment of the aneurysm. For example, without any limitation, when the expression level of a biomarker, e.g., ICAM-1, is below a reference expression level, the dosage of the aneurysm inhibitor is increased. Alternatively, the dosage of the aneurysm inhibitor can be decreased if the expression level of the biomarker is above the reference expression level.

certain embodiments, the aneurysm inhibitor can be administered once a day, twice a day, once a week, twice a week, three times a week, four times a week, five times a week, six times a week, once every two weeks, once a month, twice a month, once every other month or once every third month. In certain embodiments, the aneurysm inhibitor can be administered twice a week. In certain embodiments, the aneurysm inhibitor can be administered once a week. In certain embodiments, the aneurysm inhibitor can be administered two times a week for about four weeks and then administered once a week for the remaining duration of the treatment.

In certain embodiments, one or more aneurysm inhibitors can be used alone or in combination with one or more secondary aneurysm inhibitors. For example, but not by way of limitation, methods of the present disclosure can include administering one or more aneurysm inhibitors and one or more secondary aneurysm inhibitors. “In combination with,” as used herein, means that the aneurysm inhibitor and the one or more secondary aneurysm inhibitors are administered to a subject as part of a treatment regimen or plan. In certain embodiments, being used in combination does not require that the aneurysm inhibitor and one or more secondary aneurysm inhibitors are physically combined prior to administration, administered by the same route or that they be administered over the same time frame. In certain embodiments, the secondary aneurysm inhibitor is administered before an aneurysm inhibitor. In certain embodiments, the secondary aneurysm inhibitor is administered after an aneurysm inhibitor. In certain embodiments, the secondary aneurysm inhibitor is administered simultaneously with an aneurysm inhibitor.

A “secondary aneurysm inhibitor,” as used herein, can be any molecule, compound, chemical or composition that has an anti-aneurysm effect and is provided and/or administered in addition to the aneurysm inhibitors described herein. Secondary aneurysm inhibitors include, but are not limited to, anti-inflammatory, anti-NF-κB inhibitors, protease inhibitors, metalloproteinase inhibitors, mast cell degranulation inhibitors, free radical scavengers, and mineralocorticoid receptor antagonists. In certain embodiments, the secondary aneurysm inhibitors can be aspirin.

### 3. Biomarkers

In accordance with the disclosed subject matter, biomarkers that can be used in the methods disclosed are, for purpose of illustration and not for limitation, the size of endothelial cell-derived microvesicles, the number of endothelial cell-derived microvesicles, the presence and/or level of a protein in endothelial cell-derived microvesicles, and the presence and/or level of a nucleic acid or portion thereof, e.g., an mRNA, DNA, cDNA miRNA, snoRNA, scaRNA, lncRNA, or piRNA isolated from the endothelial cell-derived microvesicles. In certain embodiments, the endothelial cell-derived microvesicles are endothelial cell-derived exosomes.

In certain embodiments, the biomarker can be a pool of VE-cadherin, ICAM-1, or ECM1-expressing endothelial cell-derived microvesicles. In certain embodiments, a change (e.g., reduction) in a physical characteristic, e.g., number and/or concentration, or profile of the endothelial cell-derived microvesicles, e.g., VE-cadherin, ICAM-1 or ECM1-expressing endothelial cell-derived microvesicles, compared to a reference control is indicative of an aortic aneurysm in a subject.

In certain embodiments, the biomarker is a protein isolated from a pool of one or more isolated endothelial cell-derived microvesicles. In certain embodiments, the disclosure provides for methods for diagnosing and/or monitoring an aortic aneurysm in a subject that include isolating endothelial cell-derived microvesicles from a biological sample of the subject, isolating one or more protein biomarkers from the endothelial cell-derived microvesicles, wherein a change in the level and/or presence of the protein biomarker compared to a reference sample is an indication that the subject has an aortic aneurysm. In certain embodiments, the protein biomarker can be VE-cadherin, ICAM-1, ECM2, or ECM1.

In certain embodiments, the presence of the protein is detected using a reagent that specifically binds with the protein. For example, the reagent can be an antibody, an antibody derivative, an antigen-binding antibody fragment, and a non-antibody peptide that specifically binds the protein. In certain embodiments, the antibody or antigen-binding antibody fragment is a monoclonal antibody or antigen-binding fragment thereof, or a polyclonal antibody or antigen-binding fragment thereof. In certain embodiments, the protein biomarker can be detected by biophysical platforms such as mass spectrometry.

In certain embodiments, the biomarker is a nucleic acid, e.g., mRNA, isolated from or detected in a pool of one or more isolated endothelial cell-derived microvesicles. In certain embodiments, the disclosure provides for methods for diagnosing and/or monitoring an aortic aneurysm in a subject that include isolating endothelial cell-derived microvesicles from a biological sample of the subject, isolating one or more nucleic acids biomarkers from the endothelial cell-derived microvesicles, wherein a change in the level and/or presence of the nucleic acid biomarker compared to a reference sample is an indication that the subject has an aortic aneurysm. In certain embodiments, the nucleic acid biomarker can be a nucleic acid that encodes VE-cadherin, ICAM-1, ECM2, or ECM1.

In certain embodiments, detecting a transcribed polynucleotide includes amplifying the transcribed polynucleotide. In certain embodiments, the nucleic acid biomarker can be detected by RT-PCR, microarray analysis, or Q-PCR.

In addition, as outlined in detail in the Examples disclosed below, multiple proteins (Table 1) and nucleotides (Table 2) of endothelial cell-derived exosomes that are differentially expressed in subjects with the aortic disease are exemplary biomarkers that can be used in the methods disclosed.

TABLE 1
Proteomic profiles of VE-cadherin bound exosomes in MFS patients and control patients.

| Protein (gene name) | Marfan | Control |
|---|---|---|
| Alpha-2-macroglobulin (A2M) | 59 | 0 |
| Fibronectin; Anastellin; Ugl-Y1; Ugl-Y2; Ugl-Y3 | 56 | 1 |
| (FN1; DKFZp686O12165; DKFZp686L11144; DKFZp686O13149) | | |
| Complement C4-B; Complement C4 beta chain; Complement C4-B alpha | 39 | 0 |
| chain; C4a anaphylatoxin; C4b-B; C4d-B; Complement C4 gamma | | |
| chain; Complement C4-A; Complement C4 beta chain; Complement C4-A | | |
| alpha chain; C4a anaphylatoxin; C4b-A; C4d-A; Complement C4 gamma chain | | |
| (C4B; C4A) | | |
| Apolipoprotein B-100; Apolipoprotein B-48 (APOB) | 76 | 2 |
| Filamin-A (FLNA; FLJ00119) | 35 | 0 |
| Talin-1 (TLN1) | 30 | 0 |
| Haptoglobin-related protein (HPR) | 27 | 0 |
| Fibrinogen beta chain; Fibrinopeptide B; Fibrinogen beta chain (HEL-S- | 22 | 0 |
| 78p; FGB) | | |
| Vitamin K-dependent protein S (PROS1) | 22 | 0 |
| Fibrinogen gamma chain (FGG; DKFZp779N0926) | 17 | 0 |
| Complement factor H (hCG_40889; CFH) | 16 | 0 |
| Fibrinogen alpha chain; Fibrinopeptide A; Fibrinogen alpha chain (FGA) | 16 | 0 |
| Complement C3; Complement C3 beta chain; C3-beta-c; Complement C3 alpha | 15 | 0 |
| chain; C3a anaphylatoxin; Acylation stimulating protein; Complement C3b | | |
| alpha chain; Complement C3c alpha chain fragment 1 Complement C3dg | | |
| fragment; Complement C3g fragment; Complement C3d | | |
| fragment; Complement C3f fragment; Complement C3c alpha chain fragment | | |
| 2 (HEL-S-62p; C3) | | |
| Myosin-9 MYH9 | 14 | 0 |
| Apolipoprotein A-I; Proapolipoprotein A-I; Truncated apolipoprotein A-I | 14 | 0 |
| (APOA1) | | |
| CD5 antigen-like (CD5L) | 14 | 0 |
| Leucine-rich repeat-containing protein 15 (LRRC15) | 13 | 0 |
| Hemoglobin subunit beta; LVV-hemorphin-7; Spinorphin (HBB) | 11 | 1 |
| Vinculin (VCL; HEL114) | 10 | 0 |
| ATP synthase subunit beta; ATP synthase subunit beta, mitochondrial | 10 | 0 |
| (ATP5B; HEL-S-271) | | |
| Myosin regulatory light polypeptide 9 (MYL9) | 10 | 0 |
| Tubulin beta-1 chain (TUBB1) | 10 | 0 |
| Thrombospondin-1 (THBS1) | 9 | 0 |
| Actin, cytoplasmic 2; Actin, cytoplasmic 2, N-terminally processed; Actin, | 18 | 2 |
| cytoplasmic 1; Actin, cytoplasmic 1, N-terminally processed | | |
| (PS1TP5BP1; ACTG1; ACTB) | | |
| Haptoglobin; Haptoglobin alpha chain; Haptoglobin beta chain (HP) | 8 | 0 |
| Coagulation factor XIII A chain (F13A1) | 8 | 0 |
| Apolipoprotein L1 (APOL1) | 7 | 0 |
| Integrin alpha-IIb; Integrin alpha-IIb heavy chain; Integrin alpha-IIb light | 7 | 0 |
| chain, form 1; Integrin alpha-IIb light chain, form 2 (ITGA2B) | | |
| Ficolin-2 (FCN2) | 7 | 0 |
| Immunoglobulin J chain (IGJ) | 7 | 0 |
| C4b-binding protein alpha chain (C4BPA) | 33 | 5 |
| Complement C1r subcomponent; Complement C1r subcomponent heavy | 6 | 0 |
| chain; Complement C1r subcomponent light chain (C1R) | | |
| Elongation factor 1-alpha; Putative elongation factor 1-alpha-like | 6 | 0 |
| 3; Elongation factor 1-alpha 1; Elongation factor 1-alpha 2 | | |
| (EEF1A1L14; EEF1A1; EEF1A1P5; PTI-1; EEF1A2) | | |
| Apolipoprotein A-II; Proapolipoprotein A-II; Truncated apolipoprotein A-II | 6 | 0 |
| (APOA2) | | |
| Pyruvate kinase; Pyruvate kinase PKM (HEL-S-30; PKM; PKM2) | 6 | 0 |
| ATP synthase subunit alpha; ATP synthase subunit alpha, mitochondrial | 6 | 0 |
| (HEL-S-123m; ATP5A1) | | |
| Complement C1q subcomponent subunit B (C1QB) | 5 | 0 |
| Integrin-linked protein kinase (HEL-S-28; ILK) | 5 | 0 |
| Band 3 anion transport protein (SLC4A1) | 5 | 0 |
| Pleckstrin (PLEK) | 5 | 0 |
| Clusterin; Clusterin beta chain; Clusterin alpha chain; Clusterin (CLU) | 5 | 0 |
| Lipopolysaccharide-binding protein (LBP) | 5 | 0 |
| Peroxiredoxin-6 (HEL-S-128m; PRDX6) | 5 | 0 |
| Ras suppressor protein 1 (RSU1) | 5 | 0 |
| Apolipoprotein E (APOE) | 9 | 2 |
| Erythrocyte band 7 integral membrane protein (STOM) | 4 | 0 |
| Syntaxin-binding protein 2 (ZNF14; STXBP2) | 4 | 0 |
| Serum amyloid A-4 protein (SAA4) | 4 | 0 |
| Coagulation factor V; Coagulation factor V heavy chain; Coagulation factor V | 4 | 0 |
| light chain (F5) | | |
| Ig gamma-2 chain C region (DKFZp686I04196; IGHG2; DKFZp686E23209) | 4 | 0 |
| Serum deprivation-response protein (SDPR) | 4 | 0 |
| Inter-alpha-trypsin inhibitor heavy chain H1 (ITIH1) | 4 | 0 |
| 14-3-3 protein zeta/delta (YWHAZ; 14-3-3 protein) | 4 | 0 |
| Platelet glycoprotein IX (GP9) | 4 | 0 |
| Heat shock cognate 71 kDa protein; Heat shock-related 70 kDa protein 2 | 4 | 0 |
| (HSPA8; HEL-S-72p; HSPA2) | | |
| C4b-binding protein beta chain (C4BPB) | 4 | 0 |
| Tubulin beta-4B chain; Tubulin beta-3 chain; Tubulin beta chain; Tubulin beta- | 4 | 0 |
| 4A chain; Tubulin beta-2B chain; Tubulin beta-2A chain | | |
| (TUBB2C; TUBB4B; TUBB3; TUBB; TUBB4A; XTP3TPATP1; TUBB2B; | | |
| TUBB6; TUBB2A) | | |
| Keratin-associated protein 13-2 (KRTAP13-2) | 4 | 0 |
| Hemoglobin subunit alpha (HBA2; HBA1) | 8 | 2 |
| Apolipoprotein C-III (APOC3) | 7 | 2 |
| Histone H2A; Histone H2A type 1-J; Histone H2A type 1-H; Histone | 3 | 0 |
| H2A.J; Histone H2A type 1-C; Histone H2A type 3; Histone H2A type 1- | | |
| D; Histone H2A type 1; Histone H2A type 1-B/E; | | |
| Ras-related protein Rab-6A; Ras-related protein Rab-6B; Ras-related protein | 3 | 0 |
| Rab-39A (RAB6B; RAB6A; RAB39A) | | |
| Ras-related protein Rab-11A; Ras-related protein Rab-11B | 3 | 0 |
| (RAB11A; RAB11B) | | |
| Citrate synthase; Citrate synthase, mitochondrial CS | 3 | 0 |
| Epiplakin (EPPK1) | 3 | 0 |
| IgGFc-binding protein (FCGBP) | 3 | 0 |
| Ig mu chain C region; Ig mu heavy chain disease protein (IGHM) | 3 | 0 |
| Superoxide dismutase; Superoxide dismutase [Mn], mitochondrial (SOD20 | 3 | 0 |
| Tripeptidyl-peptidase 1 (TPP1) | 3 | 0 |
| Apolipoprotein C-IV (APOC4) | 3 | 0 |
| Peptidyl-prolyl cis-trans isomerase; Peptidyl-prolyl cis-trans isomerase | 3 | 0 |
| A; Peptidyl-prolyl cis-trans isomerase A, N-terminally processed (PPIA; HEL- | | |
| S-69p) | | |
| Integrin beta; Integrin beta-3 (ITGB3) | 3 | 0 |
| Myosin light polypeptide 6 (PDE6H; MYL6) | 3 | 0 |
| Fructose-bisphosphate aldolase A (ALDOA; HEL-S-87p) | 3 | 0 |
| ADP/ATP translocase 2; ADP/ATP translocase 2, N-terminally | 3 | 0 |
| processed; ADP/ATP translocase 3; ADP/ATP translocase 3, N-terminally | | |
| processed; ADP/ATP translocase 1 (SLC25A5; SLC25A4; SLC25A6) | | |
| 14-3-3 protein sigma (SFN) | 3 | 0 |
| Transgelin-2 (TAGLN2) | 3 | 0 |
| ATP synthase subunit O, mitochondrial (ATP5O) | 3 | 0 |
| V-set and immunoglobulin domain-containing protein 8 (VSIG8) | 3 | 0 |
| Fermitin family homolog 3 (FERMT3) | 3 | 0 |
| Keratin-associated protein 13-1 (KRTAP13-1) | 3 | 0 |
| Fatty acid-binding protein, epidermal (FABP5) | 3 | 1 |
| Galectin-3-binding protein (LGALS3BP) | 14 | 5 |
| Apolipoprotein C-II (APOC2; APOC4-APOC2) | 8 | 3 |
| Ig gamma-3 chain C region (IGHG3; FLJ00385; DKFZp686I15212) | 8 | 3 |
| Apolipoprotein C-I; Truncated apolipoprotein (C-IAPOC1) | 2 | 0 |
| Alpha-enolase; Enolase (ENO1; EDARADD) | 2 | 0 |
| Coronin; Coronin-1A (CORO1A) | 2 | 0 |
| Alpha-actinin-1; Alpha-actinin-4 (ACTN1; ACTN4) | 2 | 0 |
| Alpha-1-antitrypsin; Short peptide from AAT (SERPINA1) | 2 | 0 |
| Complement C1q subcomponent subunit C (C1QC) | 2 | 0 |
| Complement C1q subcomponent subunit A (C1QA) | 2 | 0 |
| Ras-related protein Rap-1b; Ras-related protein Rap-1A; Ras-related protein | 2 | 0 |
| Rap-1b-like protein (RAP1A; RAP1B) | | |
| Inter-alpha-trypsin inhibitor heavy chain H2 (ITIH2) | 2 | 0 |
| Apolipoprotein (LPA) | 2 | 0 |
| Beta-parvin (PARVB) | 2 | 0 |
| Thromboxane-A synthase (TBXAS1; hCG_14925) | 2 | 0 |
| Tropomyosin alpha-4 chain; Tropomyosin beta chain (TPM4; HEL-S- | 2 | 0 |
| 108; HEL-S-273; TPM2; TPM2b) | | |
| Serum amyloid A protein | 2 | 0 |
| Adenylyl cyclase-associated protein; Adenylyl cyclase-associated protein 1 | 2 | 0 |
| (CAP1) | | |
| Serotransferrin (TF) | 2 | 0 |
| Beta-2-glycoprotein 1 (APOH) | 2 | 0 |
| Isocitrate dehydrogenase [NADP]; Isocitrate dehydrogenase [NADP], | 2 | 0 |
| mitochondrial (IDH2) | | |
| Triosephosphate isomerase (TPI1; HEL-S-49) | 2 | 0 |
| Prohibitin-2 (PHB2) | 2 | 0 |
| Multimerin-1; Platelet glycoprotein Ia*; 155 kDa platelet multimerin | 2 | 0 |
| (MMRN1) | | |
| Cofilin-2; Cofilin-1 (CFL1; HEL-S-15; CFL2) | 2 | 0 |
| Actin-related protein 2/3 complex subunit 4 (ARPC4-TTLL3; ARPC4) | 2 | 0 |
| Cytochrome c oxidase subunit 5A, mitochondrial (COX5A) | 2 | 0 |
| Profilin-1 (PFN1) | 2 | 0 |
| Myosin regulatory light chain 12A; Myosin regulatory light chain 12B | 2 | 0 |
| (MYL12A; MYL12B) | | |
| Dual specificity protein phosphatase 14 (DUSP14) | 2 | 0 |
| Protein AMBP; Alpha-1-microglobulin; Inter-alpha-trypsin inhibitor light | 2 | 0 |
| chain; Trypstatin (AMBP) | | |
| von Willebrand factor; von Willebrand antigen 2 (VWF) | 2 | 0 |
| Galectin-7 (LGALS7) | 2 | 0 |
| Elongation factor Tu, mitochondrial TUFM | 2 | 0 |
| Keratin-associated protein 3-1 (KRTAP3-1) | 2 | 0 |
| Neutrophil defensin 3; HP 3-56; Neutrophil defensin 2; Neutrophil defensin | 2 | 1 |
| 1; HP1-56; Neutrophil defensin 2 (DEFA3; DEFA1) | | |
| Keratinocyte proline-rich protein (KPRP) | 2 | 1 |
| N-alpha-acetyltransferase 25, NatB auxiliary subunit (NAA25) | 6 | 3 |
| Ig kappa chain V-IV region Len | 6 | 3 |
| Protein arginine N-methyltransferase 5; Protein arginine N-methyltransferase | 0 | 2 |
| 5; Protein arginine N-methyltransferase 5, N-terminally processed (PRMT5) | | |
| Caspase-14; Caspase-14 subunit p19; Caspase-14 subunit p10 (CASP14) | 0 | 2 |
| Calmodulin-like protein 5 (CALML5) | 0 | 2 |
| Annexin; Annexin A2; Putative annexin A2-like protein (ANXA2; HEL-S- | 1 | 2 |
| 270; ANXA2P2) | | |
| Desmoglein-1 (DSG1) | 2 | 4 |
| Cullin-2 (CUL2) | 0 | 3 |
| Desmocollin-1 (DSC1) | 0 | 3 |
| Filaggrin-2 (FLG2) | 1 | 3 |
| Arginase-1 (ARG1) | 0 | 5 |
| SerpinB12 (SERPINB12) | 0 | 6 |
| Protein-glutamine gamma-glutamyltransferase E; Protein-glutamine gamma- | 0 | 9 |
| glutamyltransferase E 50 kDa catalytic chain; Protein-glutamine gamma- | | |
| glutamyltransferase E 27 kDa non-catalytic chain (TGM3) | | |
| Hornerin (HRNR) | 1 | 12 |
| Methylosome protein 50 (WDR77) | 1 | 16 |

TABLE 2
Microarray data for microRNA profiles for MFS plasma
versus control plasma for VE-cadherin bound exosomes.

| | Expression | Expression | |
|---|---|---|---|
| miRNA | Marfan | human control | Fold-Change |
| hsa-miR-4270 | 142.1 | 2.2 | 64.2 |
| hsa-miR-4749-5p | 146.1 | 2.4 | 60.4 |
| hsa-miR-4257 | 79.6 | 1.6 | 49.1 |
| hsa-miR-4695-5p | 73.6 | 2.5 | 29.6 |
| hsa-miR-4433b-3p | 63.3 | 2.4 | 26.2 |
| hsa-miR-3141 | 80.5 | 3.1 | 25.6 |
| hsa-miR-3656 | 150.9 | 6.2 | 24.3 |
| hsa-miR-6087 | 989.5 | 41.6 | 23.8 |
| hsa-miR-6800-5p | 36.3 | 1.6 | 22.6 |
| hsa-miR-4687-3p | 55.6 | 2.5 | 22.0 |
| hsa-miR-6743-5p | 169.2 | 8.4 | 20.1 |
| hsa-miR-6812-5p | 41.8 | 2.3 | 18.4 |
| hsa-miR-4728-5p | 40.6 | 2.3 | 17.7 |
| hsa-miR-149-3p | 73.0 | 4.2 | 17.4 |
| hsa-miR-6786-5p | 49.2 | 2.9 | 17.2 |
| hsa-miR-6858-5p | 53.9 | 3.4 | 16.0 |
| hsa-miR-328-5p | 32.9 | 2.2 | 15.2 |
| hsa-let-7b-5p | 73.9 | 5.0 | 14.9 |
| hsa-miR-4463 | 30.2 | 2.2 | 13.6 |
| hsa-miR-8060 | 35.8 | 2.7 | 13.2 |
| hsa-miR-4739 | 32.0 | 2.7 | 11.8 |
| hsa-miR-92a-3p | 22.9 | 1.9 | 11.8 |
| hsa-let-7a-5p | 23.3 | 2.0 | 11.7 |
| hsa-miR-6795-5p | 29.0 | 2.5 | 11.5 |
| hsa-miR-6769b-5p | 24.4 | 2.2 | 11.2 |
| hsa-miR-7704 | 79.1 | 7.4 | 10.6 |
| hsa-miR-7107-5p | 18.6 | 1.8 | 10.5 |
| hsa-miR-6089 | 267.4 | 26.4 | 10.1 |
| hsa-miR-4726-5p | 19.0 | 1.9 | 10.1 |
| hsa-miR-4689 | 18.6 | 1.9 | 9.6 |
| hsa-miR-8069 | 80.0 | 8.4 | 9.6 |
| hsa-miR-6787-5p | 109.6 | 11.9 | 9.2 |
| hsa-miR-6134 | 17.4 | 1.9 | 9.1 |
| hsa-miR-6724-5p | 31.9 | 3.5 | 9.1 |
| hsa-miR-6775-5p | 31.8 | 3.5 | 9.0 |
| hsa-miR-6816-5p | 19.8 | 2.2 | 9.0 |
| hsa-miR-4651 | 31.6 | 3.5 | 9.0 |
| hsa-miR-23a-3p | 29.9 | 3.4 | 8.9 |
| hsa-miR-6891-5p | 19.3 | 2.2 | 8.8 |
| hsa-miR-4488 | 33.9 | 3.9 | 8.6 |
| hsa-miR-1237-5p | 64.9 | 7.6 | 8.6 |
| hsa-miR-6879-5p | 32.9 | 3.9 | 8.4 |
| hsa-miR-6090 | 269.8 | 33.4 | 8.1 |
| hsa-miR-3196 | 43.3 | 5.4 | 8.0 |
| hsa-miR-3162-5p | 13.4 | 1.7 | 8.0 |
| hsa-miR-4529-3p | 16.5 | 2.1 | 8.0 |
| hsa-miR-6769a-5p | 18.5 | 2.4 | 7.8 |
| hsa-miR-8089 | 14.9 | 1.9 | 7.8 |
| hsa-miR-4459 | 17.0 | 2.2 | 7.8 |
| hsa-miR-1914-3p | 21.4 | 2.8 | 7.7 |
| hsa-miR-4433-3p | 17.3 | 2.2 | 7.7 |
| hsa-miR-6765-5p | 23.9 | 3.2 | 7.4 |
| hsa-miR-1915-3p | 65.4 | 9.0 | 7.3 |
| hsa-miR-6819-5p | 15.3 | 2.2 | 7.1 |
| hsa-miR-1273g-3p | 17.3 | 2.5 | 6.9 |
| hsa-miR-7111-5p | 13.1 | 1.9 | 6.9 |
| hsa-miR-6797-5p | 16.2 | 2.4 | 6.8 |
| hsa-miR-4516 | 23.7 | 3.5 | 6.8 |
| hsa-miR-4656 | 18.1 | 2.7 | 6.6 |
| hsa-let-7d-5p | 14.3 | 2.2 | 6.6 |
| hsa-miR-26a-5p | 15.4 | 2.4 | 6.3 |
| hsa-mir-6800 | 20.1 | 3.2 | 6.3 |
| hsa-miR-4497 | 18.9 | 3.0 | 6.2 |
| hsa-let-7e-5p | 11.5 | 1.8 | 6.2 |
| hsa-miR-191-5p | 16.4 | 2.6 | 6.2 |
| hsa-miR-6749-5p | 14.9 | 2.5 | 6.1 |
| hsa-miR-1207-5p | 15.9 | 2.6 | 6.0 |
| hsa-miR-103a-3p | 16.9 | 2.8 | 6.0 |
| hsa-miR-6165 | 8.9 | 1.5 | 6.0 |
| hsa-miR-6752-5p | 27.0 | 4.5 | 6.0 |
| hsa-miR-204-3p | 15.0 | 2.6 | 5.8 |
| hsa-miR-1227-5p | 21.6 | 3.8 | 5.7 |
| hsa-miR-4632-5p | 21.9 | 3.9 | 5.6 |
| hsa-miR-1228-5p | 33.6 | 6.2 | 5.5 |
| hsa-miR-3960 | 327.7 | 60.1 | 5.5 |
| hsa-miR-4732-5p | 13.4 | 2.5 | 5.4 |
| hsa-miR-6771-5p | 13.4 | 2.5 | 5.3 |
| hsa-miR-24-3p | 10.0 | 1.9 | 5.2 |
| hsa-miR-4281 | 13.1 | 2.5 | 5.2 |
| hsa-miR-5189-5p | 14.9 | 2.9 | 5.2 |
| hsa-miR-4688 | 10.8 | 2.1 | 5.0 |
| hsa-miR-6127 | 11.6 | 2.3 | 5.0 |
| hsa-miR-4655-5p | 14.4 | 2.9 | 5.0 |

The present disclosure discovers that endothelial cell-derived microvesicles (e.g. exosomes) express a number of endothelial cell-derived markers, e.g., VE-cadherin, which allow for endothelial cell-derived characterization of exosomes, and purification, isolation, and identification of endothelial cell-derived exosomes in a sample. The signal or level of the endothelial cell-derived markers detected in the same can indicate the size and/or the number of endothelial cell-derived microvesicles in the sample, and thus as biomarkers for predicting, detecting, screening, and monitoring the aortic aneurysm diseases.

Non-limiting limiting examples of endothelial cell-derived markers include VE-cadherin, vascular cell adhesion protein 1 (VCAM-1), pathologische anatomie Leiden-endothelium (PAL-E), intercellular adhesion molecule 2 (ICAM2), vascular endothelial growth factor receptor 1 (VEGFR1), multimerin 2 (EndoGlyx-1), Endoglin (cd105), cluster of differentiation 146 (CD146), intercellular adhesion molecule 1 (ICAM1), extracellular matrix protein 1 (ECM1), extracellular matrix protein 2 (ECM2) and cluster of differentiation 31 (CD31). In certain embodiments, the biomarker is selected from the group consisting of VE-cadherin, ICAM-1, E-cadherin, endothelial nitric oxide synthetase, ECM1, ECM2, and combinations thereof.

Biomarkers used in the methods of the present disclosure can be identified in a biological sample using any method known in the art. Biomarkers can be microvesicles and/or nucleic acids and/or proteins that reside on the surface or within the microvesicles. The microvesicles, e.g., exosomes, can be isolated from a biological sample and analyzed using any method known in the art. The nucleic acid sequences, fragments thereof, and proteins, and fragments thereof, can be isolated and/or identified in a biological sample using any method known in the art.

### 4. Microvesicle Isolation Techniques

Circulating tissue derived microvesicles can be isolated from a subject by techniques known in the art. Circulating tissue derived microvesicles can be isolated from a biological sample obtained from a subject, such as a blood sample, or other biological fluid. In certain embodiments, the microvesicles can be exosomes.

There are several capture and enrichment platforms that are known in the art and currently available. For example, microvesicles can be isolated by a method of differential centrifugation as described by Raposo et al. Journal of Experimental Medicine 183.3 (1996): 1161-1172. Additional methods include anion exchange and/or gel permeation chromatography as described in U.S. Pat. Nos. 6,899,863 and 6,812,023. Methods of sucrose density gradients or Organelle electrophoresis are described in U.S. Pat. No. 7,198,923. A method of magnetic activated cell sorting (MACS) is described in Taylor and Cicek Gercel-Taylor, Gynecologic oncology 110.1 (2008): 13-21. A method of nanomembrane ultrafiltration concentrator is described in Cheruvanky, et al., American Journal of Physiology-Renal Physiology 292.5 (2007): F1657-F1661. Microvesicles can be identified and isolated from a biological sample of a subject by a microchip technology that uses a unique microfluidic platform to efficiently and selectively separate microvesicles (Nagrath et al., Nature 450.7173 (2007): 1235-1239). This can be adapted to identify and separate microvesicles using similar principles of capture and separation.

The microvesicles, including exosomes, can be isolated from a biological sample and analyzed using any method known in the art. In certain non-limiting embodiments, high exclusion limit agarose-based gel chromatography can be utilized to isolate microvesicles from a recipient's plasma (Taylor et al., 2005). For example, and not by way of limitation, to isolate the total vesicle fraction, the plasma sample can be fractionated using a size exclusion column, e.g., a 2.5×30cm Sepharose 2B column run isocratically with PBS, where the elution can be monitored by absorbance at 280 nm. The fractions comprising microvesicles can be concentrated using ultrafiltration with a 100K Dalton cut-off membrane. The fractions can then be ultracentrifuged, e.g., at 120,000 g for 2 hours at 4° C. to obtain a pellet that contains microvesicles.

For immunosorbent isolation of tissue derived microvesicle populations, plasma microvesicles can be selectively incubated with antibodies specific for a microvesicle surface protein (e.g., VE-cadherin) coupled with magnetic microbeads. After incubation for 2 hours at 4° C., the magnetic bead complexes can be placed in the separator's magnetic field and the unbound microvesicles can be removed with the supernatant. The bound tissue-specific microvesicle subsets can be recovered and diluted in IgG elution buffer (Pierce Chemical Co), centrifuged and resuspended in PBS. Additional techniques based on the size and surface protein of the microvesicles can be used to isolate microvesicles, e.g., ultracentrifugation, sucrose gradient-based ultracentrifugation, ExoQuick™ (System Bioscience), and the Exo-Flow™ (System Bioscience). Tissue-specific microvesicle number and size distribution can be determined using the NanoSight NS300.

In certain embodiments, endothelial cell-derived microvesicles can be purified, isolated, and/or identified by the detection of a cell-specific marker, e.g., endothelial cell specific protein. For example, but not by way of limitation, endothelial cell-derived microvesicles can be isolated and/or identified based on the proteins residing on the surface of the microvesicles. In certain embodiments, the marker can be nucleic acids and/or proteins that reside on the surface or within the microvesicles. Non-limiting examples of such markers include VE-cadherin, ICAM-1, ECM1, ECM2, E-cadherin, endothelial nitric oxide synthetase. In certain embodiments, the cell-specific marker can be VE-cadherin, ICAM-1, ECM2, or ECM1.

In certain embodiments, a method for the isolation, identification, and/or purification of endothelial cell-derived microvesicles can include: (a) obtaining a biological sample from the subject; and (b) isolating, purifying, and/or identifying one or more endothelial cell-derived microvesicles from the biological sample by the detection of a marker specific for endothelial cell, e.g., VE-cadherin, ICAM-1, ECM1, ECM2, E-cadherin, and/or endothelial nitric oxide synthetase.

In certain embodiments, a method for the isolation, identification, and/or purification of endothelial cell-derived microvesicles can include: (a) obtaining a biological sample from the subject; (b) isolating and/or purifying one or more microvesicles from the biological sample; and (c) isolating, purifying and/or identifying one or more endothelial cell-derived microvesicles from the one or more microvesicles of (b) by detecting a marker specific for endothelial cells, e.g., VE-cadherin, ICAM-1, ECM1, ECM2, E-cadherin, and/or endothelial nitric oxide synthetase.

### 5. Protein Detection Techniques

In certain embodiments, the biomarker is a protein, present on the surface and/or within tissue-specific isolated microvesicles, e.g., exosomes. Proteins can be isolated from a microvesicle using any number of methods, which are well-known in the art, the particular isolation procedure chosen being appropriate for the particular biological sample.

Methods for the detection of protein biomarkers are well known to those skilled in the art and include but are not limited to mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation and immunohistochemistry. These methods use antibodies, or antibody equivalents, to detect protein. Antibody arrays or protein chips can also be employed, see for example U.S. Patent Application Nos: 20030013208A1; 20020155493A1, 20030017515 and U.S. Pat. Nos. 6,329,209 and 6,365,418, herein incorporated by reference in their entirety.

ELISA and RIA procedures can be conducted such that a biomarker standard is labeled (with a radioisotope such as 125I or 35S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker in the sample is allowed to react with the corresponding immobilized antibody, radioisotope, or enzyme-labeled anti-biomarker antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting the mixture with a labeled antibody. Other conventional methods can also be employed as suitable.

In certain embodiments, the method for measuring biomarker expression includes contacting a biological sample, e.g., blood, with an antibody or variant (e.g., fragment) thereof which selectively binds the biomarker, and detecting whether the antibody or variant thereof is bound to the sample. The method can further include contacting the sample with a second antibody, e.g., a labeled antibody. The method can further include one or more washing procedures, e.g., to remove one or more reagents.

It can be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene can provide a suitable support.

Enzymes employable for labeling are not particularly limited, but can be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

Other techniques can be used to detect a biomarker according to a practitioner's preference based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Antibodies (unlabeled) are then brought into contact with the support and assayed by a secondary immunological reagent, such as labeled protein A or anti-immunoglobulin (suitable labels including 125I, horseradish peroxidase, and alkaline phosphatase). Chromatographic detection can also be used.

Other machine or autoimaging systems can also be used to measure immunostaining results for the biomarker. As used herein, “quantitative” immunohistochemistry refers to an automated method of scanning and scoring samples that have undergone immunohistochemistry, to identify and quantitate the presence of a specified biomarker, such as an antigen or other protein. The score given to the sample is a numerical representation of the intensity of the immunohistochemical staining of the sample and represents the amount of target biomarker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2, or 3).

Various automated sample processing, scanning, and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Another method that can be used for detecting and quantitating biomarker protein levels is Western blotting. Immunodetection can be performed with antibody to a biomarker using the enhanced chemiluminescence system (e.g., from PerkinElmer Life Sciences, Boston, Mass.). The membrane can then be stripped and re-blotted with a control antibody, e.g., anti-actin (A-2066) polyclonal antibody from Sigma (St. Louis, Mo.).

Antibodies against biomarkers can also be used for imaging purposes, for example, to detect the presence of a biomarker in cells of a subject. Suitable labels include radioisotopes, iodine (125I, 121I), carbon (14C), sulfur (35S), tritium (3H), indium (112In), and technetium (99mTc), fluorescent labels, such as fluorescein and rhodamine and biotin. Immunoenzymatic interactions can be visualized using different enzymes such as peroxidase, alkaline phosphatase, or different chromogens such as DAB, AEC or Fast Red.

For in vivo imaging purposes, antibodies are not detectable, as such, from outside the body, and so must be labeled, or otherwise modified, to permit detection. Markers for this purpose can be any that do not substantially interfere with the antibody binding, but which allow external detection. Suitable markers can include those that can be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which can be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99 m.

The labeled antibody or antibody fragment will then preferentially accumulate at the location of cells which include a biomarker. The labeled antibody or variant thereof, e.g., antibody fragment, can then be detected using known techniques. Antibodies include any antibody, whether natural or synthetic, full length or a fragment thereof, monoclonal or polyclonal, that binds sufficiently strongly and specifically to the biomarker to be detected. An antibody can have a Kd of at most about 10-6M, 10-7M, 10-8M, 10-9M, 10-10M, 10-11M, 10-12M. The phrase “specifically binds” refers to binding of, for example, an antibody to an epitope or antigen or antigenic determinant in such a manner that binding can be displaced or competed with a second preparation of identical or similar epitope, antigen or antigenic determinant.

Antibodies and derivatives thereof that can be used encompasses polyclonal or monoclonal antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a biomarker, or portions thereof, including, but not limited to Fv, Fab, Fab′ and F(ab′)2 fragments can be used. Such fragments can be produced by enzymatic cleavage or by recombinant techniques. For example, papain or pepsin cleavage can generate Fab or F(ab′)2 fragments, respectively. Other proteases with the requisite substrate specificity can also be used to generate Fab or F(ab′)2 fragments. Antibodies can also be produced in a variety of truncated forms using antibody genes in which one or more stop codons have been introduced upstream of the natural stop site. For example, a chimeric gene encoding a F(ab′)2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 Bl; Winter, U.S. Pat. No. 5,225,539; Winter, European Patent No. 0,239,400 B1; Queen et al., European Patent No. 0451216 B1; and Padlan, E. A. et al., EP 0519596 Al. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

In certain embodiments, agents that specifically bind to a polypeptide other than antibodies are used, such as peptides. Peptides that specifically bind can be identified by any techniques known in the art, e.g., peptide phage display libraries. Generally, an agent that is capable of detecting a biomarker polypeptide, such that the presence of a biomarker is detected and/or quantitated, can be used. As defined herein, an “agent” refers to a substance that is capable of identifying or detecting a biomarker in a biological sample (e.g., identifies or detects the mRNA of a biomarker, the DNA of a biomarker, the protein of a biomarker). In one embodiment, the agent is a labeled or labelable antibody which specifically binds to a biomarker polypeptide.

In addition, a biomarker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution including a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide techniques for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

For additional information regarding mass spectrometers, see, e.g., Principles of Instrumental Analysis, 3rd edition. Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of a particular biomarker. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in certain embodiments a control sample can include heavy atoms (e.g., 13C) thereby permitting the test sample to be mixed with the known control sample in the same mass spectrometry run.

In certain embodiments, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In certain embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard including a known amount of a biomolecule can be analyzed as the second sample to better quantify the amount of the biomolecule present in the first sample. In certain embodiments, the identity of the biomolecules in the first and second samples can also be determined.

### 6. RNA Detection Techniques

In certain embodiments, the biomarker is a nucleic acid, including DNA and/or RNA (e.g., mRNA), contained within the tissue-specific isolated microvesicles, e.g., exosomes. In certain embodiments, the biomarker is a miRNA. In certain embodiments, the biomarker is an mRNA. Nucleic acid molecules can be isolated from a microvesicle using any number of methods, which are well-known in the art, the particular isolation procedure chosen being appropriate for the particular biological sample. Examples of methods for extraction are provided in the Examples section herein. In certain instances, with some techniques, it may also be possible to analyze the nucleic acid without extraction from the microvesicle.

In certain embodiments, the analysis of nucleic acids present in the microvesicles is quantitative and/or qualitative. Any method for qualitatively or quantitatively detecting a nucleic acid biomarker can be used. Detection of RNA transcripts can be achieved, for example, by Northern blotting, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using amplification methods. For example, it is within the scope of the present disclosure to reverse transcribe mRNA into cDNA followed by polymerase chain reaction (RT-PCR); or, to use a single enzyme for both as described in U.S. Pat. No. 5,322,770, or reverse transcribe mRNA into cDNA followed by symmetric gap ligase chain reaction (RT-AGLCR) as described by R. L. Marshall et al., PCR Methods and Applications 4: 80-84 (1994).

In certain embodiments, quantitative real-time polymerase chain reaction (qRT-PCR) is used to evaluate RNA levels of biomarker. The levels of a biomarker and a control RNA can be quantitated in cancer tissue or cells and adjacent benign tissues. In certain embodiments, the levels of one or more biomarkers can be quantitated in a biological sample.

Other known amplification methods which can be utilized herein include, but are not limited to, the so-called “NASBA” or “3SR” technique described in PNAS USA 87: 1874-1878 (1990) and also described in Compton, Nature 350 (1991): 91-92;); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO9322461.

In situ hybridization visualization can also be employed. Another method for detecting mRNAs in a microvesicle sample is to detect mRNA levels of a marker by fluorescent in situ hybridization (FISH). FISH is a technique that can directly identify a specific sequence of DNA or RNA in a cell, microvesicle sample or biological sample and therefore enables to visual determination of the marker presence and/or expression in tissue samples. Fluorescence in situ hybridization is a direct in situ technique that is relatively rapid and sensitive. FISH test also can be automated. Immunohistochemistry can be combined with a FISH method when the expression level of the marker is difficult to determine by immunohistochemistry alone.

Alternatively, RNA expression can be detected on a DNA array, chip or a microarray. Oligonucleotides corresponding to the biomarker(s) are immobilized on a chip which is then hybridized with labeled nucleic acids of a test sample obtained from a subject. Positive hybridization signal is obtained with the sample including biomarker transcripts. Methods of preparing DNA arrays and their use are well known in the art. (See, for example, U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To detect RNA molecules, for example, mRNA can be extracted from the microvesicle sample to be tested, reverse transcribed and fluorescent-labeled cDNA probes are generated.

Using microarrays capable of hybridizing to a marker, cDNA can be probed with the labeled cDNA probes, and they can be slides scanned and the fluorescence intensity measured. This intensity correlates with the hybridization intensity and expression levels.

Types of probes for detection of RNA include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the particular biomarker RNA. The probes can be as short as is required to differentially recognize the particular biomarker RNA transcripts, and can be as short as, for example, 15 bases; however, probes of at least 17 bases, e.g., 18 bases or better 20 bases can be used. In certain embodiments, the primers and probes hybridize specifically under stringent conditions to a nucleic acid fragment having the nucleotide sequence corresponding to the target gene. As herein used, the term “stringent conditions” means hybridization will occur only if there is at least 95% and at least 97% identity between the sequences.

The form of labeling of the probes can be any that is appropriate, such as the use of radioisotopes, for example, 32P and 35S. Labeling with radioisotopes can be achieved, whether the probe is synthesized chemically or biologically, by the use of suitably labeled bases.

Exemplary probes and primers that can be used in the methods of the present disclosure are presented below.

### 7. Kits

The present disclosure further provides kits for diagnosing and/or monitoring a subject with an aortic aneurysm that provides for isolating, purifying and/or detecting one or more endothelial cell-derived microvesicles. In certain embodiments, the kit can include one or more provisions for detecting one or more markers, e.g., biomarkers, present on the surface of the endothelial cell-derived microvesicles or present within the endothelial cell-derived microvesicles. In certain embodiments, a kit of the present disclosure can further include one or more markers for isolating microvesicles from a biological sample. The disclosure further provides for kits for assessing the efficacy of a therapeutic treatment regime of a subject having aortic aneurysm disease.

Types of kits include, but are not limited to, packaged probe and primer sets (e.g., TaqMan probe/primer sets), arrays/microarrays, endothelial cell-specific antibodies and antibody-conjugated beads or quantum dots, which further contain one or more probes, primers or other detection reagents for isolating and/or detecting one or more microvesicles and/or one or more endothelial cell-derived microvesicles, disclosed herein. For example, but not by way of limitation, the endothelial cell marker can include VE-cadherin, ICAM-1, E-cadherin, ECM1, ECM2, endothelial nitric oxide synthetase. In certain embodiments, the endothelial cell marker can be VE-cadherin, ICAM-1 or ECM1.

In certain non-limiting embodiments, a kit can include a pair of oligonucleotide primers suitable for polymerase chain reaction (PCR) or nucleic acid sequencing, for detecting one or more biomarker(s) to be identified. A pair of primers can include nucleotide sequences complementary to a biomarker and be of sufficient length to selectively hybridize with said biomarker. Alternatively, the complementary nucleotides can selectively hybridize to a specific region in close enough proximity 5′ and/or 3′ to the biomarker position to perform PCR and/or sequencing. Multiple biomarker-specific primers can be included in the kit to simultaneously assay large number of biomarkers. The kit can also include one or more polymerases, reverse transcriptase and nucleotide bases, wherein the nucleotide bases can be further detectably labeled.

In certain non-limiting embodiments, a primer can be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length.

In certain non-limiting embodiments, the oligonucleotide primers can be immobilized on a solid surface or support, for example, on a nucleic acid microarray, wherein the position of each oligonucleotide primer bound to the solid surface or support is known and identifiable.

In certain non-limiting embodiments, a kit can include at least one nucleic acid probe, suitable for in situ hybridization or fluorescent in situ hybridization, for detecting the biomarker(s) to be identified. Such kits will generally include one or more oligonucleotide probes that have specificity for various biomarkers.

In certain non-limiting embodiments, a kit can include at least one antibody for immunodetection of the biomarker(s) to be identified. Antibodies, both polyclonal and monoclonal, specific for a biomarker, can be prepared using conventional immunization techniques, as will be generally known to those of skill in the art. The immunodetection reagents of the kit can include detectable labels that are associated with, or linked to, the given antibody or antigen itself. Such detectable labels include, for example, chemiluminescent or fluorescent molecules (rhodamine, fluorescein, green fluorescent protein, luciferase, Cy3, Cy5, or ROX), radiolabels (3H, 35S, 32P, 14C, 131I) or enzymes (alkaline phosphatase, horseradish peroxidase).

In certain non-limiting embodiments, the biomarker-specific antibody can be provided bound to a solid support, such as a column matrix, an array, or well of a microtiter plate. Alternatively, the support can be provided as a separate element of the kit.

In certain non-limiting embodiments, a kit can include one or more primers, probes, microarrays, or antibodies suitable for detecting one or more biomarkers.

In certain non-limiting embodiments, where the measurement techniques in the kit employ an array, the set of biomarkers set forth above can constitute at least 10 percent or at least 20 percent or at least 30 percent or at least 40 percent or at least 50 percent or at least 60 percent or at least 70 percent or at least 80 percent of the species of markers represented on the microarray.

In certain non-limiting embodiments, a biomarker detection kit can include one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction to detect a biomarker. A kit can also include additional components or reagents necessary for the detection of a biomarker, such as secondary antibodies for use in immunohistochemistry.

In certain non-limiting embodiments, a biomarker detection kit can include one or more reagents and/or tools for isolating endothelial cell-derived microvesicles from a biological sample. A kit can also include reagents necessary for isolating the protein and/or nucleic acids from the isolated microvesicles.

A kit can further include techniques for comparing the biomarker with a reference standard and can include instructions for using the kit to detect the biomarker of interest. In certain embodiments, the instructions describe that the change in the level and/or presence of a biomarker, set forth herein, indicates that the subject is developing or had aortic aneurysm disease.

### 8. Exemplary Embodiments

The present disclosure relates to biomarker for predicting, diagnosing, and or monitoring an aortic aneurysm disease in a subject. The present disclosure provides methods for predicting, diagnosing, or monitoring aortic aneurysm disease in a subject, including determining the presence and/or level of a biomarker in a biological sample (e.g., a blood sample) obtained from a subject. The present disclosure further provides methods for determining the responsive to an aortic aneurysm disease treatment in a patient having the aortic aneurysm disease. The presently disclosed methods can also provide for early prognosis and diagnosis of aortic aneurysm disease (e.g., identification of a biomarker prior to the onset of a disease).

In certain embodiments, the aortic aneurysm disease is an abdominal aortic aneurysm disease, an ascending aortic aneurysm disease, a descending aortic aneurysm disease, or combinations thereof. In certain embodiments, the aortic aneurysm disease is Marfan Syndrome (MFS) disease.

In one aspect, the present disclosure provides a method for diagnosing a subject with an aortic aneurysm. The method includes obtaining a biological sample from the subject, detecting and/or isolating one or more biomarkers from the biological sample, and diagnosing aortic aneurysm in the subject when there is a change in the presence and/or level of the one or more biomarkers.

In another aspect, the present disclosure provides a method for diagnosing a subject with an aortic aneurysm. The method includes obtaining a biological sample from a subject, isolating, purifying and/or identifying one or more endothelial cell-derived microvesicles from the biological sample, analyzing one or more biomarkers associated with the endothelial cell-derived microvesicles, and diagnosing the subject with an aortic aneurysm when there is a change in the presence and/or level of the one or more biomarkers as compared to a reference control level.

In a further aspect, the present disclosure provides a method for assessing the efficacy of a therapy for treating aortic aneurysm disease in a subject. The method includes determining the level of one or more biomarker in a biological sample obtained from the subject prior to therapy, and determining the level of the one or more biomarkers in a biological sample obtained from the subject, at one or more points during therapy, wherein the therapy is efficacious for treating aortic aneurysm in the subject when there is a change in the level of the one or more biomarkers in the second or subsequent samples, relative to the first sample.

In certain embodiments, the biomarker is a protein. In certain embodiments, the biomarker is a nucleic acid. In certain embodiments, the biomarker is selected from the group consisting of VE-cadherin, ICAM-1, ECM1, ECM2 and combinations thereof. In certain embodiments, a reduction in the level of a VE-cadherin biomarker compared to a reference control is indicative that the subject has an aortic aneurysm. In certain embodiments, a reduction in the level of an ICAM-1 biomarker compared to a reference control is indicative that the subject has an aortic aneurysm. In certain embodiments, a reduction in the level of an ECM1 biomarker compared to a reference control is indicative that the subject has an aortic aneurysm. In certain embodiments, a reduction in the level of an ECM2 biomarker compared to a reference control is indicative that the subject has an aortic aneurysm. In certain embodiments, a change in the level of the biomarker indicates a change of the size of the aortic aneurysm.

The present disclosure further provides a method for diagnosing a subject with an aortic aneurysm. The method includes obtaining a biological sample from a subject, isolating, purifying and/or identifying one or more endothelial cell-derived microvesicles from the biological sample, analyzing the number of endothelial cell-derived microvesicles expressing a endothelial cell specific protein, and diagnosing the subject with an aortic aneurysm when the number of expressing a endothelial cell specific protein-expressing endothelial cell-derived microvesicles is reduced compared to a reference control.

In another aspect, the present disclosure provides a method for treating a subject with aneurysm. The method includes obtaining a biological sample from a subject, isolating, purifying and/or identifying one or more endothelial cell-derived microvesicles from the biological sample, analyzing the number of endothelial cell-derived microvesicles expressing a endothelial cell specific protein, diagnosing the subject with an aortic aneurysm when the number of expressing a endothelial cell specific protein-expressing endothelial cell-derived microvesicles is reduced compared to a reference control, and treating the subject diagnosed with the aortic aneurysm.

In certain embodiments, treating the subject diagnosed with an aneurysm comprises administration of a beta blocker. In certain embodiments, the endothelial cell specific protein is selected from the group consisting of VE-cadherin, ICAM-1, E-cadherin, endothelial nitric oxide synthetase, ECM1, ECM2 and combinations thereof.

In a further aspect, the present disclosure provides a method for isolating, purifying or identifying endothelial cell-derived microvesicles from a biological sample. The method includes obtaining a biological sample from a subject, isolating and/or purifying one or more microvesicles from the biological sample, and isolating, purifying and/or identifying one or more endothelial cell-derived microvesicles from the one or more isolated and/or purified microvesicles by detecting a marker specific for endothelial cells. In certain embodiments, the marker specific for endothelial cells is a protein. In certain embodiments, the protein is a surface protein. In certain embodiments, the marker specific for endothelial cell is selected from the group consisting of VE-cadherin, ICAM-1, E-cadherin, endothelial nitric oxide synthetase, ECM1, ECM2 and combinations thereof.

In certain embodiments, the subject is human. In certain embodiments, the biological sample is a blood sample. In certain embodiments, the aortic aneurysm is a descending aortic aneurysm, ascending aortic aneurysm, and/or abdominal aortic aneurysm. In certain embodiments, the subject is a Marfan syndrome patient.

The present disclosure further provides for diagnosing and/or monitoring a subject with an aortic aneurysm. For example, but not by way of limitation, the kit can include reagents useful for detecting a marker specific to an endothelial cell-derived microvesicle. In certain embodiments, the kit further includes a packaged probe and primer set, arrays/microarrays, marker-specific antibodies or marker-specific antibody-conjugated beads or quantum dots. In certain embodiments, the kit further comprises a pair of oligonucleotide primers, suitable for polymerase chain reaction or nucleic acid sequencing, for detecting the marker. In certain embodiments, the kit further comprises a monoclonal antibody or antigen-binding fragment thereof, or a polyclonal antibody or antigen-binding fragment thereof, for detecting the marker. In certain embodiments, the marker specific for endothelial cell is selected from the group consisting of VE-cadherin, ICAM-1, E-cadherin, endothelial nitric oxide synthetase, ECM1, ECM2 and combinations thereof.

### EXAMPLE

The presently disclosed subject matter will be better understood by reference to the following Example, which is provided as exemplary of the presently disclosed subject matter, and not by way of limitation.

### Example 1

### Circulating Endothelial Specific Exosomes as Noninvasive Biomarkers of Aortic Aneurysm Disease

The present Example shows that circulating endothelial specific exosome profiles were significantly altered in patients with an aortic aneurysm disease compared to age and gender matched controls. Validation by analysis of endothelial specific exosomes in a Marfan (MFS) mouse model of aortic aneurysm disease and aneurysm patients were performed. The results demonstrate that circulating endothelial exosomes enable noninvasive diagnosis of ascending aortic aneurysm disease.

The present Example examined the correlation between aortic root size with corresponding changes in the plasma endothelial exosome profiles of patients with MFS. It was found that immediate processing of plasma samples provides more reliable and consistent results, than stored samples that have been thawed several times. Mouse and human studies were performed to characterize the endothelial biomarkers, VE-cadherin and ICAM-1, in patients with various size aneurysms. Human patient samples were collected according to the

IRB protocol. Exosomes were isolated through ultracentrifugation. The exosomes were analyzed for particle size and concentration using the Nanosight nanoparticle detector prior to being loaded into a Western blot. The patient samples were age and gender matched with non-aneurysm control patients. The ages ranged from 31 to 86 years old. The patients' blots were then probed for the endothelial cell biomarkers, as well as known exosome markers. The results have been mirrored across 9 patients. The western blots were quantified using ImageQuant analysis software to compare control and aneurysm groups. Different sizes of aneurysms were recorded in the initial experiments, but not yet grouped and compared against each other. Unpaired T-test statistical tests were performed on the data to give a significant p-value for both VE-cadherin and ICAM-1 downregulation between groups.

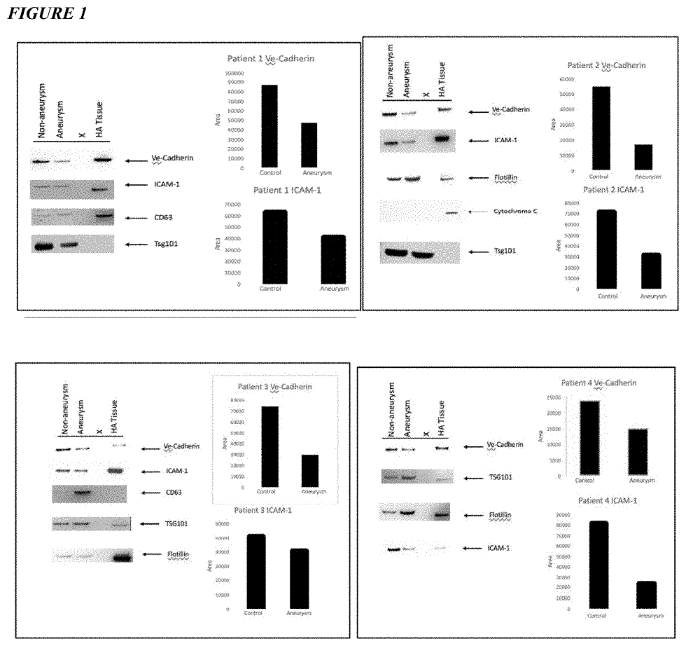

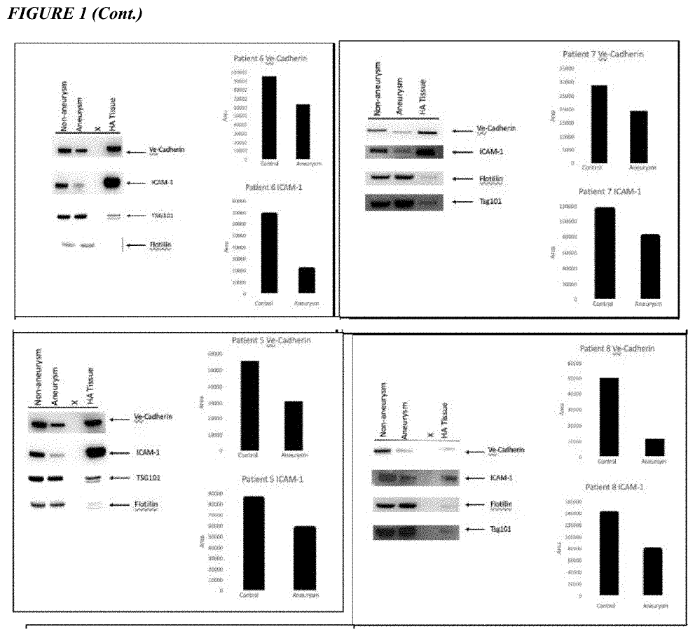

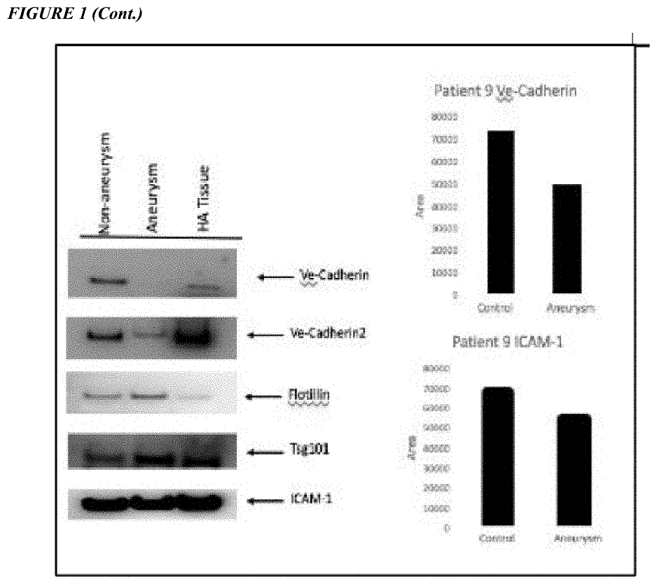

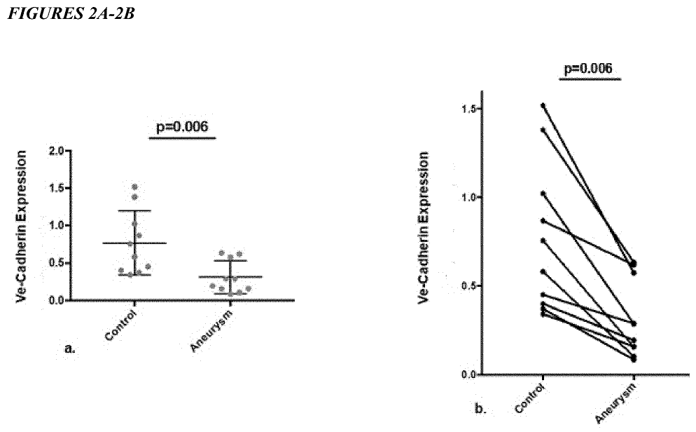

The compilation images of 9 of the patients' western blot expressions are shown in FIG. 1, with the initial non-normalized differences between control and aneurysm for both endothelial cell biomarkers. These values were normalized to TSG101 and plotted together in FIGS. 2A-2B and 3A-3B with an additional control patient and aneurysm patient (for a total of 10 patients for each group). A universal downregulation of VE-cadherin (FIGS. 2A-2B) and ICAM-1 (FIGS. 3A-3B) was observed in the aneurysm patients as compared to non-aneurysm control patients.

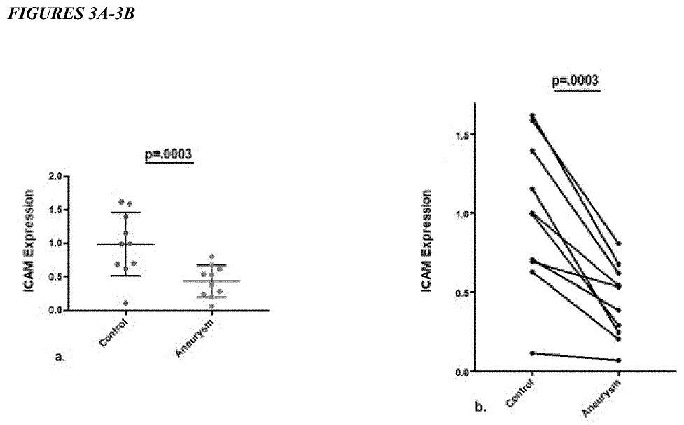

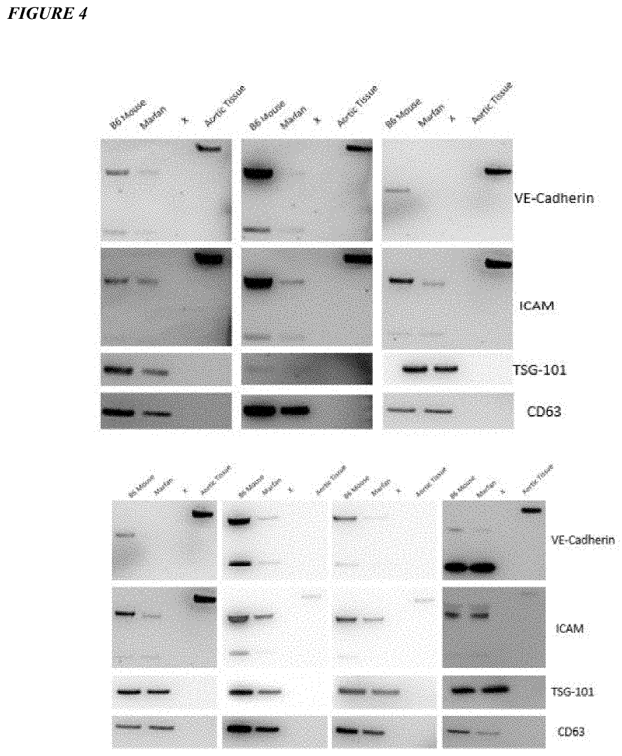

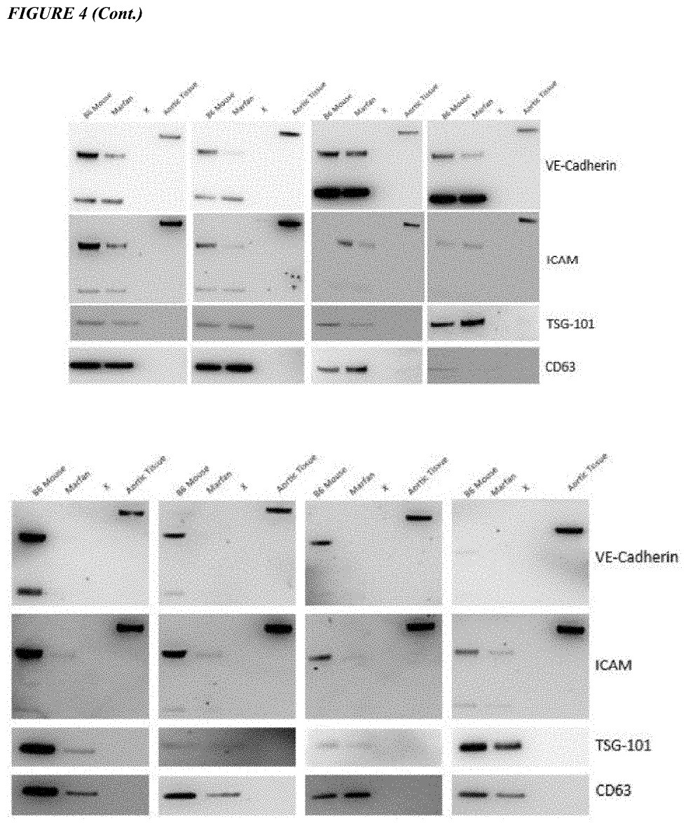

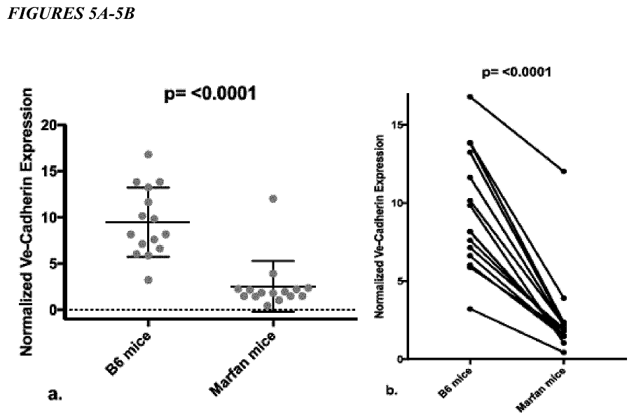

Similar results were also observed in the Marfan mouse model. The plasma of fifteen B6 mice and fifteen Marfan Syndrome induced mice were obtained. Isolation of exosomes was done through ultracentrifugation. The number of particles was detected with a nanoparticle detector (Nanosight NS 400). Through western blot analysis, various endothelial cell biomarkers were tested, such as VE-cadherin and ICAM (FIG. 4). The results consistently showed a downregulation of the respective proteins in the Marfan mice when compared to the B6 mice (FIGS. 5A-5B). Using Image Quant analysis software, these values were quantified and normalized to the TSG 101 signal. On average, the VE-cadherin signal in the B6 mouse shows a 5-fold increase compared to the Marfan samples.

The human and mouse results disclosed herein suggest the discovery of reliable endothelial biomarker. Based on the above results, more human patient samples are collected analyzing aneurysm size versus biomarker expression. Functional vasculogenic assays using human aortic endothelial cells give a representation of 3D vascular growth in Marfan patients. These results establish the association between aneurysm size and Marfan groups.

The effects of initiation of medical therapy are investigated regarding beta-blockade or angiotensin receptor blockade on endothelial exosome profiles in the newly diagnosed MFS patients. Patients with Marfan syndrome are recruited to assess whether changes in aneurysm size by imaging correlate with changes in endothelial specific exosome quantitative profiles.

Furthermore, endothelial specific exosome purification was successfully achieved. Endothelial cell specific exosomes were collected from 3 aneurysm patients and their matched controls (n=6 total) and isolated the RNA cargo. Next generation sequencing of the microRNA cargoes was performed with ingenuity pathway analysis to understand whether specific angiogenic and vascular smooth muscle pathways are specifically altered in endothelial specific exosomes from aneurysm subjects compared to the age matched controls. Hybrid mass spectrometry analysis of endothelial specific exosomes from 3 controls and aneurysm subjects were performed to understand for functional and diagnostic differences in the intraexosomal proteomic cargoes. The in vitro analysis suggests that endothelial specific exosomes have functional effects. Endothelial tube formation assay was performed on human umbilical vein endothelial cells (HUVECs) incubated with exosomes from aneurysm versus control subjects and noted significantly decreased endothelial tube formation in HUVECs incubated with aneurysm plasma exosomes.

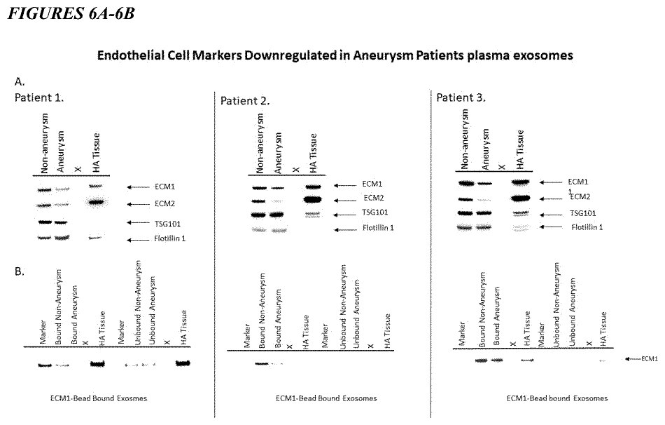

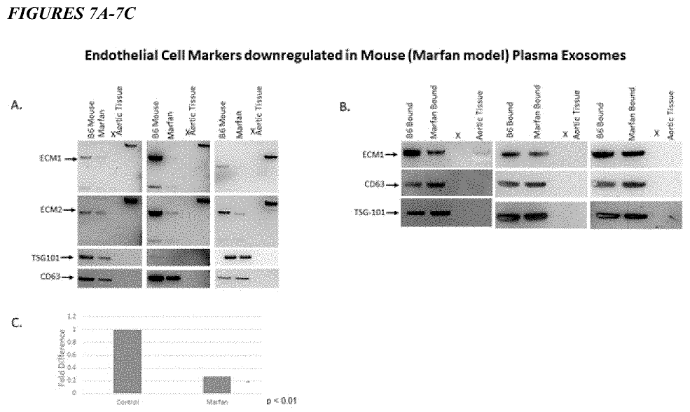

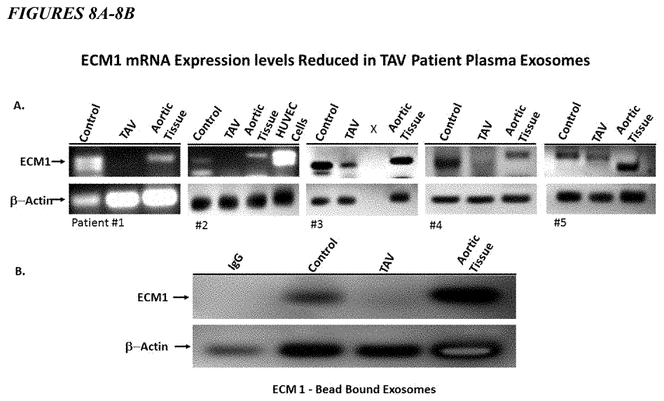

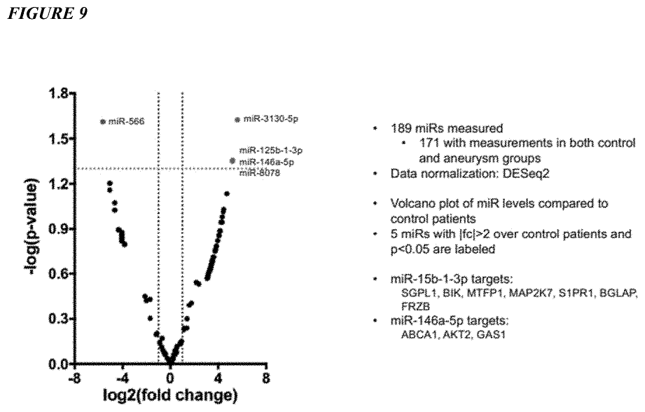

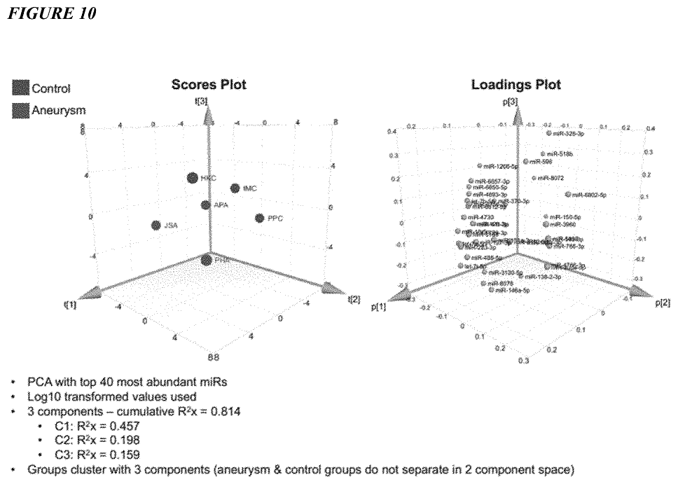

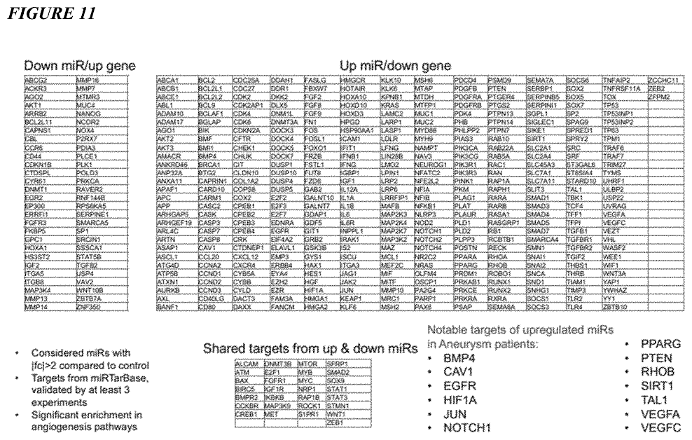

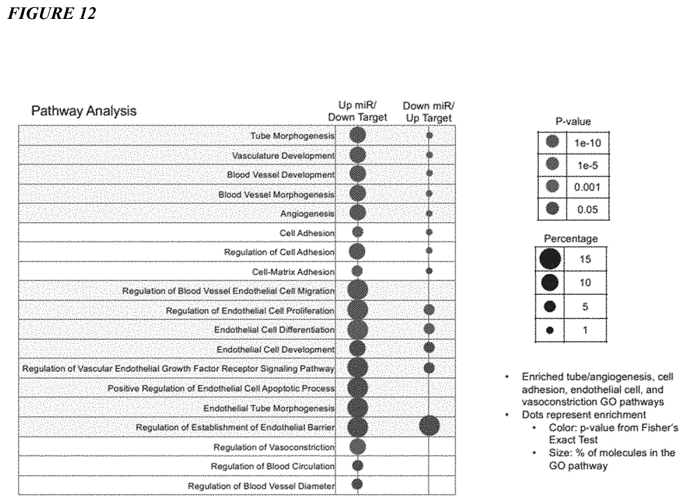

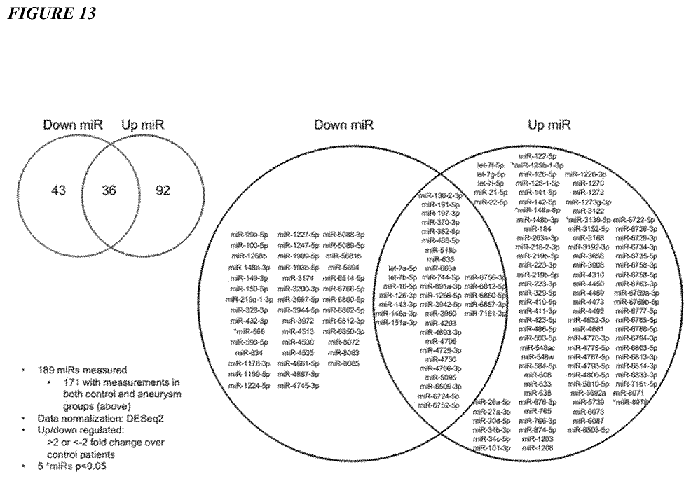

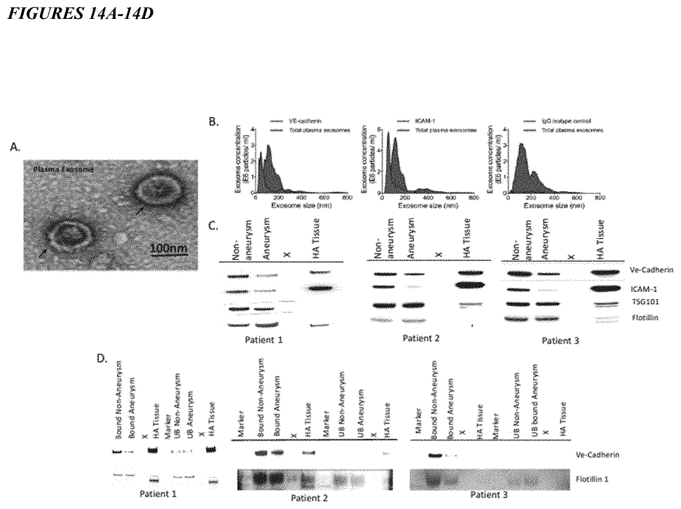

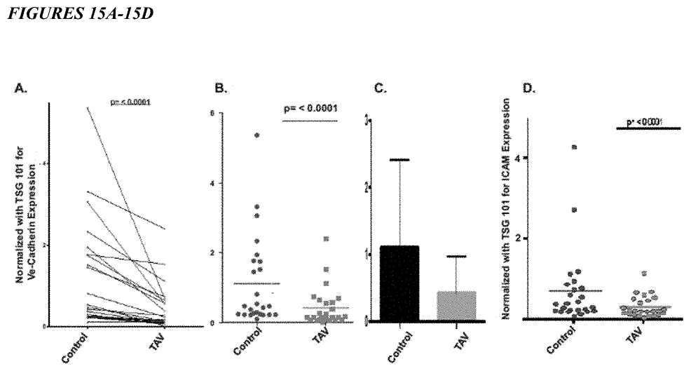

Other endothelial markers in the endothelial exosome subpopulation were also tested, including differences in expression of E-cadherin, endothelial nitric oxide synthetase, ECM1 and ECM2. As show in FIG. 6A, downregulation of ECM1 and ECM2 was observed in the aneurysm patients as compared to non-aneurysm control patients. The number of ECM1 positive endothelial cell-derived exosomes were reduced in aneurysm patients compared to control patients (FIG. 6B). Similar results were observed in Marfan syndrome mouse (FIGS. 7A-7C). Further analysis of ECM1 mRNA expression shows that the amount of ECM1 mRNA is significantly reduced in endothelial cell-derived exosomes in TAV patients as compared to control patients (FIG. 8A). In addition, ECM1 protein expression was also greatly reduced in endothelial cell-derived exosomes in TAV patients as compared to control patients (FIG. 8B).

Next, it was tested whether endothelial cell-derived exosomes from Marfan syndrome patients had an effect on the functionality of endothelial cells. Endothelial cell-derived exosomes from Marfan syndrome patients diminished the functional ability to promote angiogenesis compared to plasma exosomes from control subjects.

Understanding the expression patterns of various endothelial markers in circulating exosomes help better understanding if medical treatments for aneurysm disease lead to changes in endothelial exosome profiles. Such changes may enable the development of a noninvasive diagnostic for monitoring aneurysm disease, especially the effects of treatments on aneurysm disease. For example, the impact of medical therapy for aortic aneurysm disease on endothelial exosome profiles is examined. Whereas some patients respond to beta blocker therapy, others do not. The endothelial exosome profiles reflect the effects of medical treatment, and thus this presently disclosed subject matter can be used for selecting treatments for aortic aneurysm disease.

The present example also non-invasively examined over 100 patients with ascending aortic aneurysm disease and aneurysm disease and comparing them to age and gender matched controls. Further, this example evaluated the profiles in over 100 patients with aneurysm disease.

These results show that the present disclosure provides a reliable non-invasive technique to detect and evaluate the presence/absence and development of aneurysm and that they can be applied in conditions of aortic aneurysm disease in other anatomic locations such as descending thoracic aorta, and abdominal aorta. Overall, the presently disclosed methods allow the personalization of therapies in patients with aneurysm disease.

### Example 2

### Circulating Endothelial-Specific Exosome Profiles Enable Non-Invasive Screening for Ascending Aortic Aneurysm Disease

The present Example shows that circulating endothelial-specific exosome profiles were significantly altered in patients with ascending aortic aneurysm disease compared to age and gender-matched controls.

Plasma exosomes were isolated from 25 presurgical aneurysm patients with ascending aortic aneurysm disease, along with 25 age and gender-matched volunteer subjects who served as controls (Control group). Electron microscopic image of plasma extracellular vesicles revealed that most of the nanoparticles isolated were in the size range of exosomes (FIG. 14A). First, it was confirmed that microvesicles isolated from human plasma were enriched in exosomes, without contamination from cellular constituents/apoptotic bodies (FIG. 14C). Nanoparticle detector analysis demonstrated that majority of exosomes isolated from plasma have surface expression of VE-cadherin and ICAM-1 endothelial markers (FIG. 14B). Next, VE-cadherin was chosen as a more accurate marker with specificity for endothelial exosomes, as the former proteins were also highly expressed by platelets and leukocytes. On Western blot, lower levels of VE-cadherin and ICAM protein expression were seen in the aneurysm patient samples compared to the controls (FIG. 14C). To purify endothelial-specific exosomes (ESEs), total plasma exosomes were incubated with anti-VE-cadherin antibody-conjugated beads, and the bead-bound fraction representing ESEs was analyzed for enrichment of VE-cadherin. On Western blot, VE-cadherin bound exosomes showed enrichment of VE-cadherin compared to VE-cadherin unbound, and IgG isotype bead-bound and unbound fractions (FIG. 14D). Further, in aneurysm plasma sample decreased level of VE-cadherin protein was detected in the VE-cadherin bound exosomes on Western blot compared to the control sample. Western blots were quantified using ImageQuant analysis software to compare control and aneurysm groups. Unpaired T-test statistical tests were performed on the data to give a significant p-value for both VE-cadherin and ICAM-1 downregulation between groups. The data were normalized to TSG 101 protein for the 25 controls and aneurysm patient expression values for VE-cadherin and ICAM were connected to show a universal downregulation in the aneurysm patients (FIGS. 15A-15D). Also, 25 patients per group were categorized to show the intergroup reliability for VE-cadherin and ICAM expression (FIGS. 15A-15D). Taken together, this demonstrated that anti-VE-cadherin antibody conjugated beads can be utilized to purify a subpopulation of exosomes representing contribution from endothelial cells into the peripheral circulation

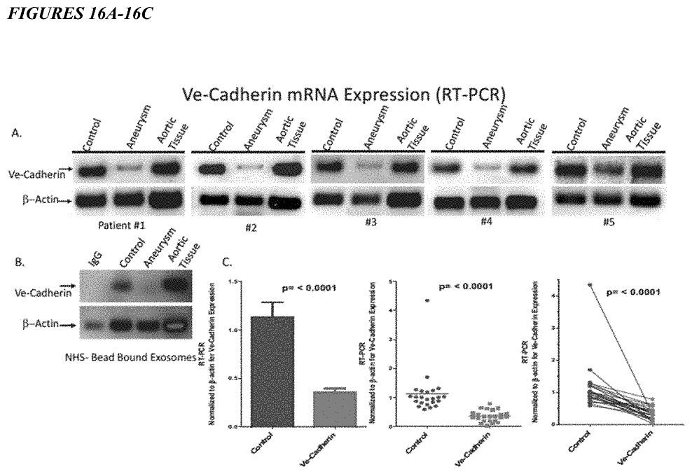

Next, it was tested whether the VE-cadherin protein expression in the plasma exosomal pool translated to differences at the mRNA level in TAV (n=25) and control (n=25) subjects. On RT-PCR analysis, TAV plasma exosomes also contained decreased levels of VE-Cadherin mRNA compared to their age and gender matched control subjects (FIG. 16A). ImageQuant analysis software was used to quantitate and compare control and aneurysm groups VE-cadherin mRNA band signals. Unpaired statistical T-test on the data normalized with TSG 101 gave a significant p-value for VE-cadherin downregulation between groups. Endogenously expressed exosomal protein TSG 101 protein was used to normalize VE-cadherin mRNA expression values for all the 25 controls and 25 aneurysm patients were connected to show a universal downregulation in the aneurysm patients (FIG. 16C). Anti-VE-cadherin antibody conjugated beads were incubated with total plasma exosomes and the bead bound fraction was analyzed for enrichment of VE-cadherin specific exosomes compared to the unbound fraction (FIG. 16B). RT-PCR analysis showed VE-cadherin bound exosomes had enrichment of VE-cadherin mRNA compared to VE-cadherin unbound, and IgG isotype bead bound and unbound fractions. In aneurysm plasma sample decreased level of VE-cadherin protein was detected in the VE-cadherin bound exosomes compared to the control sample.

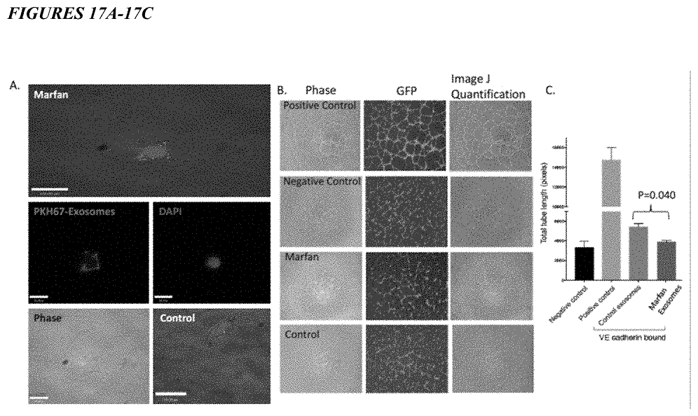

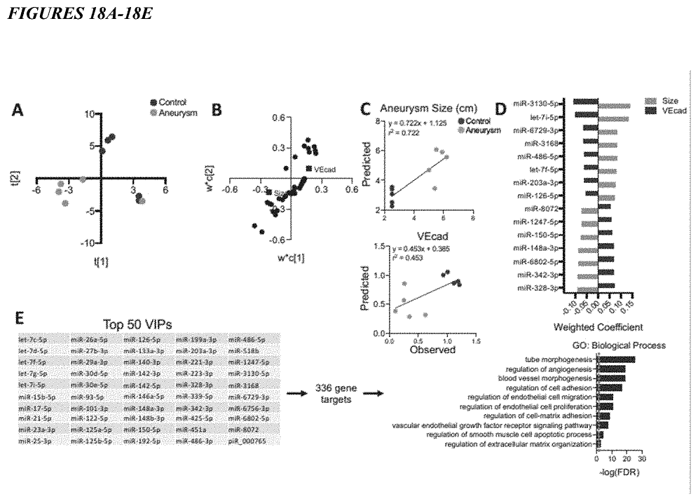

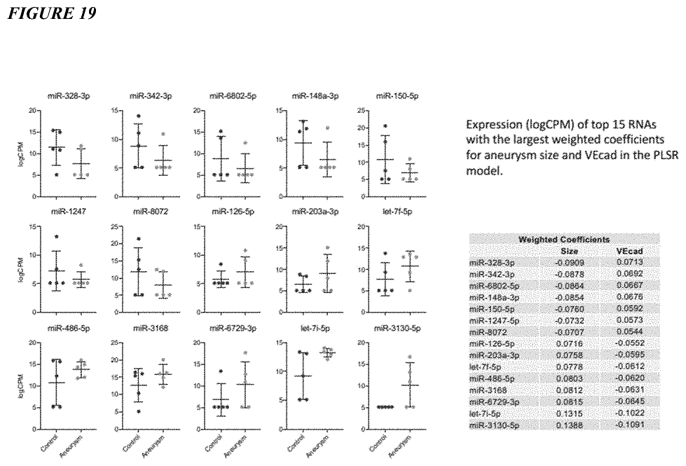

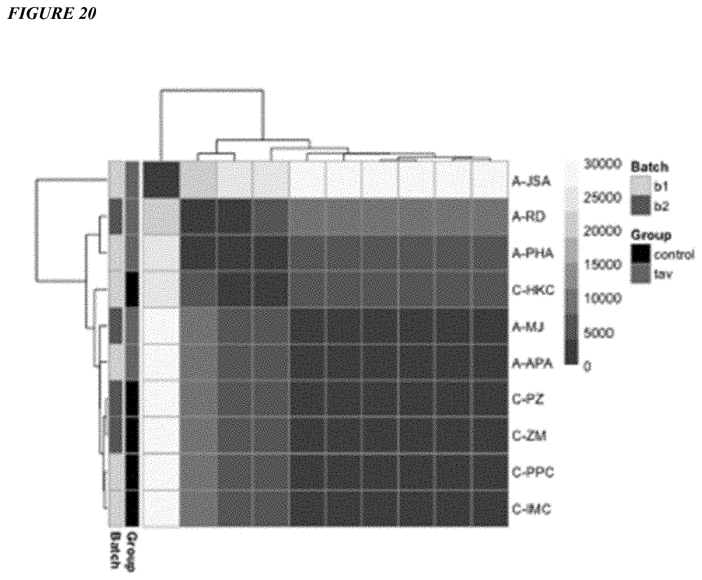

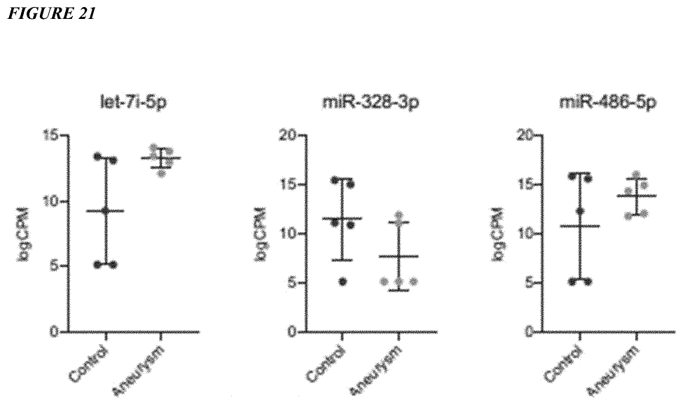

Given the results of pathway analyses for ESE cargoes from MFS sample, it was determined whether ESEs have functional effects. First, it was confirmed that ESEs were taken up by endothelial cells. To this aim, HUVECs were incubated with labeled ESEs from MFS patients (n=6) and Control subjects (n=6) and confocal microscopy pictures were taken. In both groups, HUVECs showed uptake of plasma exosomes (FIGS. 17A-17C). Next, ESEs from MFS patients (n=6) and Control subjects (n=6) were incubated with HUVECs to assess for endothelial tube formation as a marker of angiogenesis. MFS ESEs showed significantly decreased angiogenesis potential compared to control ESEs (FIGS. 18A-18E). Taken together, this suggested that ESEs from MFS patients have diminished functional ability to promote angiogenesis compared to plasma exosomes from Control subjects.

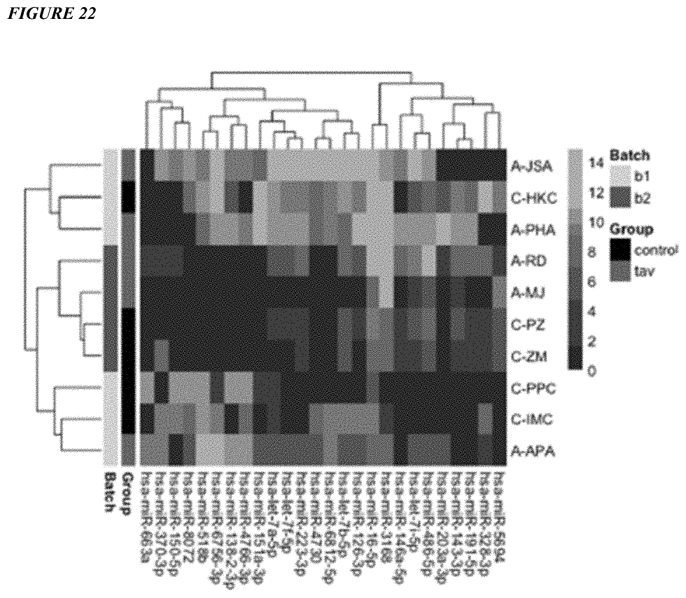

The microRNA cargoes of plasma ESE were profiled to assess for differences between the two groups. VE-cadherin antibody bound exosome fractions from 5 TAV aneurysm patients profiled the ESE cargoes using small RNA sequencing platforms. Similar ESE cargo analysis was performed on VE-cadherin antibody bound fractions from 5 Control samples. Profiling of the small RNA cargoes of TAV versus non aneurysm control samples showed that the majority of the small RNAs were microRNAs. Heatmap representation of microRNAs that were 5-fold differentially regulated between the two groups is shown in FIG. 22. Ingenuity pathway analysis showed differentially regulated microRNAs were associated with pathways involving tube morphogenesis, regulation of angiogenesis, blood vessel morphogenesis, cell adhesion, migration, apoptotic process, and vascular endothelial growth factor receptor signaling pathway (FIG. 18E).

Next, five samples from TAA patients and five control samples with age and gender matched were used to further validate the miRNAs selected from the bioinformatics analysis. Based on computational analyses, top five miRNAs that were highly expressed in aneurysm were identified and cross validated in TAA patients using quantitative PCR analysis. miR-148a-3p, miR-328-3p, and let-7i-5p were detected and TAA patients endothelial specific EVs RNA cargo showed high levels of microRNAs expression compared to control subjects endothelial specific EVs. Together, these results showed that a computational model could predict the functional potential of miRNAs.

### Example 3

### Methods

The present Example illustrates exemplary methods used for the data discussed in Example 1 and Example 2.

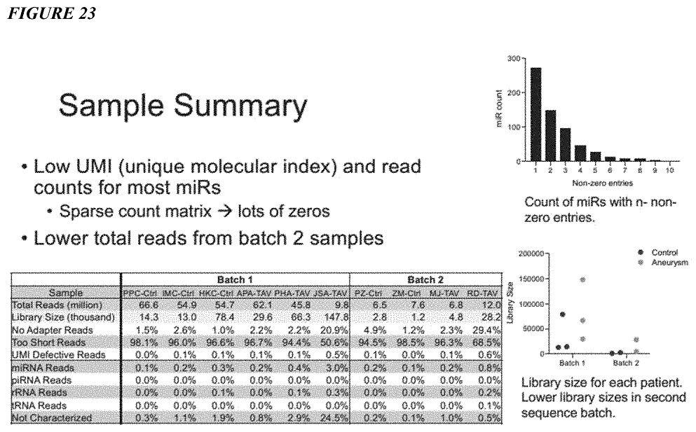

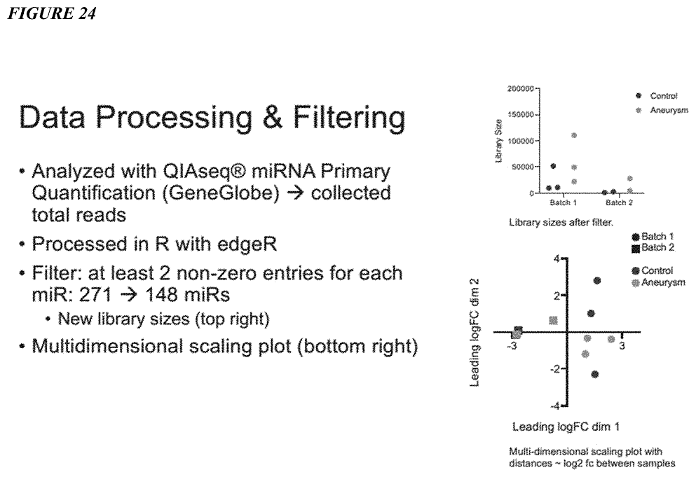

Endothelial specific exosome purification was successfully achieved from samples of patients with ascending aortic aneurysm disease or aneurysm disease and controls. Endothelial cell specific exosomes were collected, and the RNA cargo was isolated. Next generation sequencing of the microRNA cargoes was performed with ingenuity pathway analysis. Sequencing files were analyzed with QIAseq® miRNA Primary Quantification (GeneGlobe). Reads from 271 total miRNAs and piRNAs were filtered, removing RNAs with only one non-zero entry across all samples (FIGS. 23 and 24). The remaining 148 RNA reads were normalized to account for differences in sample library size using the trimmed mean of M values (TMM) method. LogCPM values were used for partial least squares regression (PLSR).

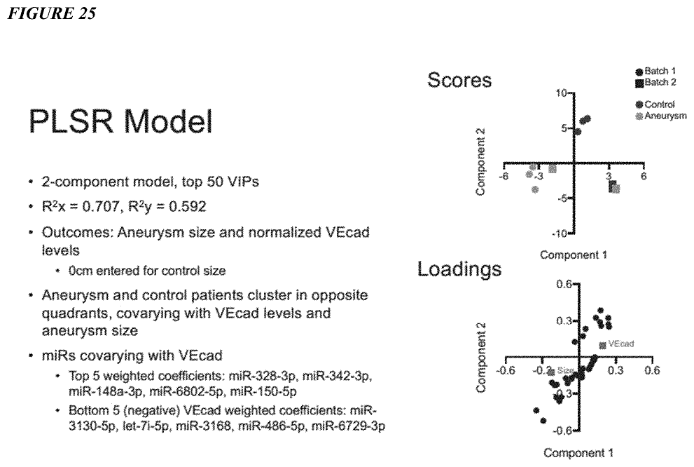

To construct the PLSR model, SIMCA-P software (Umetrics) was used to solve the PLSR problem with the nonlinear iterative partial least squares algorithm. Dimension reduction transformed the data into 2-component space, and feature selection further reduced the model to the top 50 variables important for the model projection (VIPs) (FIG. 25).

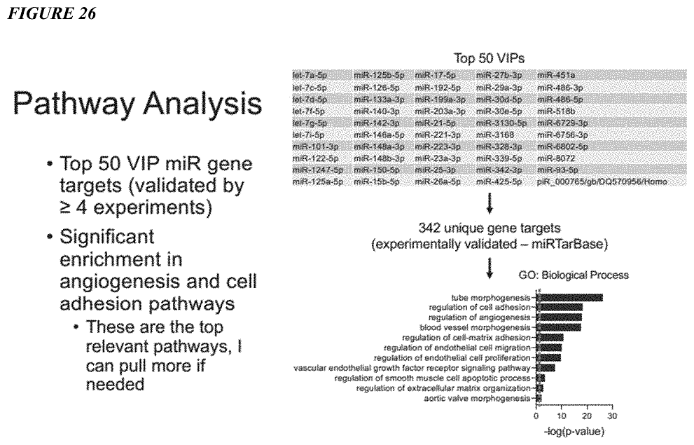

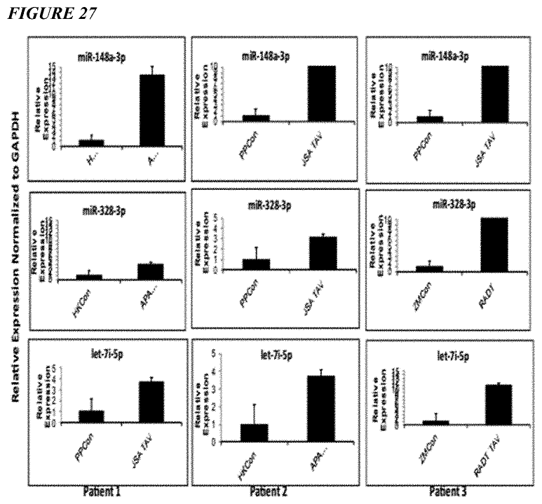

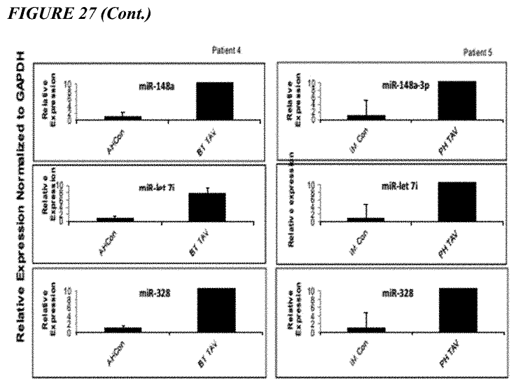

miRTarBase was used to identify miRNA gene targets (validated by at least four assays; http://mirtarbase.mbc.nctu.edu.tw). For pathway analysis, the genes were evaluated for fit to Gene Ontology (GO) Biological Process with STRING. Significantly enriched and relevant pathways were selected by false discovery rates (FDR) <0.05 (FIG. 26).

Although the presently disclosed subject matter and its advantages have been described in detail, it should be understood that various changes, substitutions and alterations can be made herein without departing from the spirit and scope of the invention. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the inventions of which are incorporated herein by reference in their entireties for all purposes.

What is claimed is:

1. A method for treating a subject with aneurysm, comprising:
(a) measuring, in a fraction of a biological sample from a subject, at least one biomarker; and
(b) administering an effective amount of an aneurysm inhibitor to the subject, when the at least one biomarker is reduced compared to a reference sample.

2. The method of claim 1, wherein the fraction is enriched with endothelial cell-derived microvesicles.

3. The method of claim 2, wherein the endothelial cell-derived microvesicles comprise an endothelial cell specific protein.

4. The method of claim 3, wherein the endothelial cell specific protein is selected from the group consisting of VE-cadherin, ICAM-1, E-cadherin, endothelial nitric oxide synthetase, ECM1, ECM2, and combinations thereof.

5. The method of claim 1, wherein the at least one biomarker is selected from the group consisting of VE-cadherin, ICAM-1, ECM1, ECM2, and combinations thereof.

6. The method of claim 1, wherein the at least one biomarker is a protein, a nucleic acid, a number of microvesicles, or combinations thereof.

7. The method of claim 1, wherein the aneurysm is an aortic aneurysm.

8. The method of claim 6, wherein the aortic aneurysm is a descending aortic aneurysm, an ascending aortic aneurysm, and/or an abdominal aortic aneurysm.

9. The method of claim 1, wherein the subject has Marfan syndrome.

10. The method of claim 1, wherein the aneurysm inhibitor is selected from the group consisting of a beta blocker, a calcium channel blocker, an angiotensin II receptor blocker, a statin, and combinations thereof.

11. The method of claim 10, wherein the beta blocker is selected from the group consisting of acebutolol, atenolol, betaxolol, bisoprolol, carteolol, labetalol, metoprolol, nadolol, nebivolol, penbutolol, pindolol, propranolol, sotanol, timolol, and combinations thereof.

12. The method of claim 10, wherein the calcium channel blocker is selected from the group consisting of amlodipine, beprifil, diltiazem, felodipine, isradipine, nicardipine, nifedipine, nisoldipine, verapamil, and combinations thereof.

13. The method of claim 10, wherein the angiotensin II receptor blocker is selected from the group consisting of azilsartan, candesartan, eprosartan, irbesartan, losartan, olmesartan, termisartan, valsartan, and combinations thereof.

14. The method of claim 10, wherein the statin is selected from the group consisting of atorvastatin, fluvastatin, lovastatin, pitavastatin, pravastatin, rosuvastatin, simvastatin, and combinations thereof.

15. A kit for diagnosing and/or monitoring a subject with an aortic aneurysm, comprising reagents for detecting a marker specific for an endothelial cell-derived microvesicle.

16. The kit of claim 15, comprising a packaged probe and primer set, arrays/microarrays, marker-specific antibodies or marker-specific antibody-conjugated beads or quantum dots.

17. The kit of claim 15, comprising a pair of oligonucleotide primers, suitable for polymerase chain reaction or nucleic acid sequencing, for detecting the marker.

18. The kit of claim 15, comprising a monoclonal antibody or antigen-binding fragment thereof, or a polyclonal antibody or antigen-binding fragment thereof, for detecting the marker.

19. The kit of claim 15, wherein the marker specific for endothelial cell is selected from the group consisting of VE-cadherin, ICAM-1, E-cadherin, endothelial nitric oxide synthetase, ECM1, ECM2 and combinations thereof.
